(19) United States
(12) Patent Application Publication
(10) Pub. No.: US 2025/0315980 A1
(43) Pub. Date: Oct. 9, 2025

(54) SYSTEMS AND METHODS FOR COMMISSIONING A MACHINE VISION SYSTEM

(71) Applicant: COGNEX CORPORATION, Natick, MA (US)

(72) Inventors: Caitlin WURZ, Natick, MA (US); Henry ATKINS, III, Natick, MA (US); Sinha TANMAY, Natick, MA (US); Deepak SURANA, Natick, MA (US); Georges GAUTHIER, Cambridge, MA (US); Ahmed EL-BARKOUKY, Hopkinton, MA (US); Patrick BRODEUR, Natick, MA (US); Patrick LUTZKE, Natick, MA (US); Jens RUETTEN, Aachen (DE); Tony DEPRE, Natick, MA (US)

(21) Appl. No.: 18/864,052

(22) PCT Filed: May 9, 2023
(86) PCT No.: PCT/US2023/066773

§ 371 Date: Nov. 8, 2024

Related U.S. Application Data
(60) Provisional application No. 63/339,912, filed on May 9, 2022.

(51) Int. Cl. G06T 7/80

(52) U.S. Cl. CPC G06T 7/80; G06T 2200/24

(57) ABSTRACT

Methods and systems are provided for commissioning machine vision systems. The methods and systems described herein may automatically configure, or otherwise assist users in configuring, a machine vision system based on a specification package.

### CROSS-REFERENCE TO RELATED APPLICATIONS

This application claims priority to U.S. Provisional Application No. 63/339,912, filed on May 9, 2022, the entire contents of which is incorporated herein by reference.

### STATEMENT REGARDING FEDERALLY SPONSORED RESEARCH

N/A

### BACKGROUND

The present technology relates to imaging systems, including machine vision systems that are configured to acquire and analyze images of objects or symbols (e.g., barcodes).

Machine vision systems are generally configured for use in capturing images of objects or symbols and analyzing the images to identify the objects or decode the symbols. Accordingly, machine vision systems generally include one or more devices for image acquisition and image processing. In some applications, these devices may be used to acquire images, or to analyze acquired images, such as for the purpose of decoding imaged symbols, such as barcodes or text. In some contexts, machine vision and other imaging systems may be used to acquire images of objects that may be larger than a field of view (“FOV”) for a corresponding imaging device and/or that may be moving relative to an imaging device.

However, prior to implementing a machine vision system (i.e., prior to performing image capture and analysis functionality), the machine vision system is commissioned (or configured), calibrated, and the like. Some approaches to commissioning a machine vision system involve individually commissioning each imaging device included in the machine vision system. As one example, a user may identify each imaging device, assign or select a name (or identifier) for each imaging device, and configure each imaging device (e.g., with a configuration and/or firmware file associated with technical settings for an imaging device). Such a process may be prone to human error, and, ultimately, inefficient commissioning of machine vision systems. As one example, some commissioning approaches lead to variability in performance of a system, divergence from planned system specifications, and increased labor costs.

The discussion above is merely provided for general background information and is not intended to be used as an aid in determining the scope of the claimed subject matter.

### SUMMARY

Accordingly, embodiments described herein provide methods and systems for commissioning machine vision systems for tunnels. Embodiments described herein automatically set-up (or configure) a machine vision system (or tunnel) based on a single specification package. In some embodiments described herein, multiple imaging devices of a machine vision system may be simultaneously configured, rather than individually configuring each imaging device. However, in some configurations, the technology disclosed herein may be implemented to automatically configure a machine vision system having a single imaging device. As such, the technology disclosed herein may be implemented for automatically configuring machine vision systems of varied complexity levels, such as complex machine vision systems including multiple imaging devices as well as less complex machine vision systems including a single imaging device.

Some embodiments described herein may utilize a stationary or moving calibration target to automatically identify and configure each imaging device included in the machine vision system. As one example, the calibration target can include a set of graphical position representations (e.g., symbols), where each graphical position representation represents a position of that graphical position representation on the stationary calibration target. Based on one or more images of the calibration target, the occurrence and arrangement of the one or more graphical position representations in the one or more images enables embodiments described herein to determine a position in space of a corresponding imaging device relative to the calibration target.

As used herein, “tunnel” may refer to a structure that supports one or more imaging devices to acquire imaging data relative to a common scene, where the scene can be a relatively small area (e.g., a table top, a discrete section of a transport system, etc.), and, within a given tunnel, there can be overlap between field of views of imaging devices, no overlap between field of views of imaging devices, or a combination thereof. Additionally, as noted herein, a tunnel may include any number of imaging devices (e.g., a single imaging device or multiple imaging devices).

One embodiment provides a method of commissioning an imaging device within a set of imaging devices of a machine vision system. The method may include receiving commissioning data including a set of identifiers. The method may also include controlling the imaging device to capture image data of a calibration target. The method may also include determining, based on the captured image data, an identifier from the set of identifiers associated with the imaging device. The method may also include configuring the imaging device based on the determined identifier and the commissioning data. The method may also include generating and transmitting a commissioning report for display to a user via a display device. The commissioning report may indicate whether the imaging device was successfully configured.

In some embodiments, receiving the commissioning data may include receiving specification data identifying the set of imaging devices associated with the machine vision system and a technical setting for each imaging device.

In some embodiments, the method may further include generating and transmitting a commissioning details user interface for display to the user via the display device, the commissioning details user interface may prompt the user to select a commissioning parameter; and receiving a set of commissioning parameters based on user input provided via the commissioning details user interface, where the set of commissioning parameters may include at least one a tunnel identifier, a site identifier, or an operator identifier.

In some embodiments, generating and transmitting the commissioning report may include generating and transmitting a commissioning report including the set of commissioning parameters.

In some embodiments, the method may further include generating and transmitting a methodology user interface for display to the user via the display device, the methodology user interface may prompt the user to select a methodology parameter; and receiving a set of methodology parameters based on user input provided via the methodology user interface. The set of methodology parameters may include at least one selected from a group consisting of a commissioning methodology, details of a calibration target (e.g., a material of the calibration target, an identifier of a particular calibration target type, or a dimension of the calibration target).

In some embodiments, determining the identifier for the imaging device may include determining the identifier for the imaging device using the set of methodology parameters.

In some embodiments, the method may further include generating and transmitting a pre-commissioning checklist user interface for display to the user via the display device, where the pre-commissioning checklist user interface may include a set of pre-commissioning tasks to be performed prior to commissioning; and receiving user confirmation that each pre-commissioning task included in the set of pre-commissioning tasks was completed.

In some embodiments, configuring the imaging device based on the identifier may include configuring the imaging device based on the identifier in response to receiving a user input confirming the association of the imaging device with the identifier.

In some embodiments, the method may further include generating and outputting an auto-naming user interface for display to the user via the display device, the auto-naming user interface may indicate an identification status for each imaging device included in the set of imaging devices.

In some embodiments, generating and outputting the auto-naming user interface may include generating and outputting an auto-naming user interface indicating that a particular imaging device of the set of imaging devices was not associated with an identifier.

In some embodiments, the method may further include receiving a user-selected identifier for the particular imaging device based on user interaction with the auto-naming user interface. The user-selected identifier may be included in a list of remaining identifiers included in the auto-naming user interface.

In some embodiments, the list of remaining identifiers may be generated based on identifiers that are not yet associated with any imaging device.

In some embodiments, each identifier of the set of identifiers may be associated with at least one imaging device of the set of imaging devices.

Another embodiment provides a system for commissioning an imaging device within a set of imaging devices for a machine vision system. The system may include at least one electronic processor. The at least one electronic processor may be configured to receive commissioning data including a set of identifiers. The at least one electronic processor may be configured to receive a set of commissioning parameters based on user input provided via a commissioning details user interface. The at least one electronic processor may be configured to receive user confirmation based on user input provided via a pre-commissioning checklist user interface. The user confirmation may confirm that each pre-commissioning task included in a set of pre-commissioning tasks was completed. The at least one electronic processor may be configured to control the imaging device to capture image data of a calibration target. The at least one electronic processor may be configured to determine, based on the captured image data, an identifier from the set of identifiers associated with the imaging device. The at least one electronic processor may be configured to configure the imaging device based on the identifier and the commissioning data. The at least one electronic processor may be configured to generate and transmit a commissioning report for display to a user via a display device. The commissioning report may indicate whether the imaging device of was successfully configured and including the set of commissioning parameters.

In some embodiments, each identifier of the set of identifiers may be associated with at least one imaging device of the set of imaging devices.

Yet another embodiment provides a method of commissioning machine vision systems for tunnels. The method may include controlling acquisition of a plurality of images, including controlling a plurality of imaging devices to cause each imaging device of the plurality of imaging devices to capture an image of a calibration object. Each of the imaging devices of the plurality of imaging devices may have a factory calibration. The method may also include determining a field calibration for each imaging device of the plurality of imaging devices based on the image acquired by the imaging device, or otherwise configuring. The method may also include determining an updated calibration for each imaging device of the plurality of imaging devices based on the factory calibration and the field calibration for the imaging device.

In some embodiments, the method may further include determining an identifier for at least one imaging device of the plurality of imaging devices based on the updated calibration.

In some embodiments, the identifier may be determined without calibrating the at least one imaging device of the plurality of imaging devices to an operational reference frame that includes the calibration object.

In some embodiments, capturing the image for each of the imaging devices of the plurality of imaging devices may include capturing the plurality of images such that the image of a first imaging device of the plurality of imaging devices and the image of a second imaging device of the plurality of imaging devices include imaging data that represents a plurality of the same features on the calibration object. The plurality of the same features may include a plurality of symbols on the calibration object that encode corresponding location information on the calibration object.

In some embodiments, the plurality of images may include a first plurality of images with the calibration object in a first location and a second plurality of images with the calibration object in a second location.

In some embodiments, the calibration object may be a first calibration object and where the image for one or more of the imaging devices of the plurality of imaging devices may include the first calibration object and a second calibration object.

In some embodiments, determining the updated calibration based on the factory calibration and the field calibration may include determining a transform between calibrations for a plurality of the imaging devices based on the image that includes the first calibration object and the second calibration object.

Yet another embodiment provides a method of commissioning machine vision systems fur tunnels. The method may include receiving, with one or more electronic processors, tunnel commissioning data. The method may also include generating, with the one or more electronic processors, a graphical user interface (GUI) including a graphical representation of a virtual tunnel representing a tunnel being commissioned for display. The method may also include receiving, with the one or more electronic processors, a first selection via the GUI, the first selection selecting an imaging device of the tunnel. The method may also include controlling, with the one or more electronic processors, an indicator of the imaging device. The method may also include receiving, with the one or more electronic processors, a second selection via the GUI. The second selection may select a virtual imaging device of the virtual tunnel. A location of the virtual imaging device on the virtual tunnel may correspond to a location of the imaging device on the tunnel. The method may also include determining, with the one or more electronic processors, a corresponding identifier for the imaging device based on the second selection. The method may also include configuring, with the one or more electronic processors, the imaging device based on the corresponding identifier and the commissioning data. The method may also include generating and transmitting, with the one or more electronic processors, a commissioning report for display to a user via a display device. The commissioning report may indicate whether the imaging device was successfully configured.

This Summary and the Abstract are provided to introduce a selection of concepts in a simplified form that are further described below in the Detailed Description. The Summary and the Abstract are not intended to identify key features or essential features of the claimed subject matter, nor are they intended to be used as an aid in determining the scope of the claimed subject matter.

### BRIEF DESCRIPTION OF THE DRAWINGS

The following drawings are provided to help illustrate various features of non-limiting examples of the disclosure and are not intended to limit the scope of the disclosure or exclude alternative implementations.

FIG. 1A schematically illustrates an example of a system for capturing multiple images of each side of an object according to some embodiments.

FIG. 1B schematically illustrates an example of a system for capturing multiple images of each side of an object according to some embodiments.

FIG. 2 schematically illustrates another example of a system for capturing multiple images of each side of an object according to some embodiments.

FIG. 3 schematically illustrates another example of a system for capturing multiple images of each side of an object according to some embodiments.

FIG. 4 schematically illustrates a system for commissioning a machine vision system of a tunnel according to some embodiments.

FIG. 5 schematically illustrates a server included in the system of FIG. 4 according to some embodiments.

FIG. 6 is a flowchart illustrating a method for commissioning a machine vision system for a tunnel using an auto-naming process for identifying one or more imaging devices associated with the tunnel using the system of FIG. 4 according to some embodiments.

FIG. 7 illustrates an example commissioning details user interface according to some embodiments.

FIG. 8 illustrates an example application details checklist user interface according to some embodiments.

FIG. 9 illustrates an example pre-commissioning checklist user interface according to some embodiments.

FIG. 10 illustrates an example methodology user interface according to some embodiments.

FIGS. 11-13 illustrate an example auto-naming user interface according to some embodiments.

FIG. 13 illustrates an example device configurations user interface according to some embodiments.

FIG. 14 illustrates an example device configuration user interface according to some configurations.

FIG. 15 illustrates an example confirmation dialogue box according to some embodiments.

FIG. 16 illustrates an example network configurations user interface according to some embodiments.

FIGS. 17-21 illustrate an example bank validation user interface according to some embodiments.

FIG. 22 is a flowchart illustrating a method for commissioning a machine vision system for a tunnel using a manual naming process for identifying one or more imaging devices associated with the tunnel according to some embodiments.

FIGS. 23-24 illustrate an example graphical user interface associated with a manual naming process according to some configurations.

FIG. 25A illustrates an example of a factory calibration setup that can be used to find a transformation between an image coordinate space and a calibration target coordinate space according to some embodiments.

FIG. 25B illustrates an example of coordinate spaces and other aspects for a calibration process, including a factory calibration and a field calibration that includes capturing images of one or more sides of an object in accordance with some embodiments.

FIG. 25C illustrates an example of a field calibration process for generating an imaging device model useable to transform coordinates of an object in a 3D coordinate space, including capturing images of one or more sides of the object in accordance with some embodiments.

FIGS. 26A-26B illustrates an example of a field calibration process associated with different positions of a calibration target (or targets) according to some embodiments.

### DETAILED DESCRIPTION OF THE PRESENT DISCLOSURE

As noted above, machine vision systems (including one or more imaging devices) are generally configured for use in capturing images of objects or symbols and analyzing the images to identify the objects or decode the symbols. Machine vision systems may vary in terms of complexity (e.g., number of imaging devices). For instance, a complex machine vision system may include multiple imaging devices while a less complex machine vision system may include a single imaging device. Accordingly, in some examples, a machine vision system may include a single imaging device. In other examples, a machine vision system may include multiple imaging devices.

However, prior to implementing a machine vision system (i.e., prior to performing image capture and analysis functionality) the machine vision system is commissioned, calibrated, and the like. Some approaches to commissioning a machine vision system may involve individually commissioning each imaging device included in the machine visions system. As one example, following some approaches, a user may identify each imaging device, assign or select a name (or identifier) for each imaging device, and configure each imaging device (e.g., with a configuration or firmware file associated with technical settings for an imaging device). Such a process is prone to human error, and, ultimately, inefficient commissioning of machine vision systems. As one example, some commissioning approaches may lead to variability in performance of a system, divergence from planned system specifications, and increased labor costs.

Accordingly, embodiments described herein provide methods and systems for commissioning machine vision systems (e.g., for tunnels) such that commissioning of machine vision systems is performed with an increased accuracy, efficiency, and the like. Embodiments described herein automatically set-up (or configure) a machine vision system (or tunnel) based on a specification package (e.g., a single specification package file, a package folder, or a package database (or database entries)). Alternatively, or in addition, embodiments described herein may utilize a stationary or moving calibration target to automatically identify and configure each imaging device included in the machine vision system. Accordingly, in some embodiments described herein, multiple imaging devices of a machine vision system may be simultaneously configured (i.e., rather than individually configuring each imaging device). However, in some configurations, the technology disclosed herein may be implemented to automatically configure a machine vision system having a single imaging device. As such, the technology disclosed herein may be implemented for automatically configuring machine vision systems of varied complexity levels, including machine vision systems having a single imaging device and machine vision systems having multiple imaging devices.

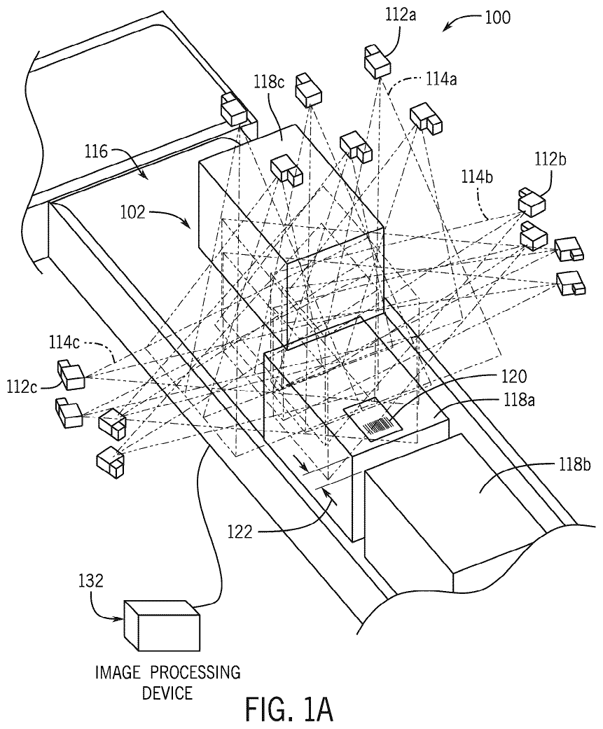

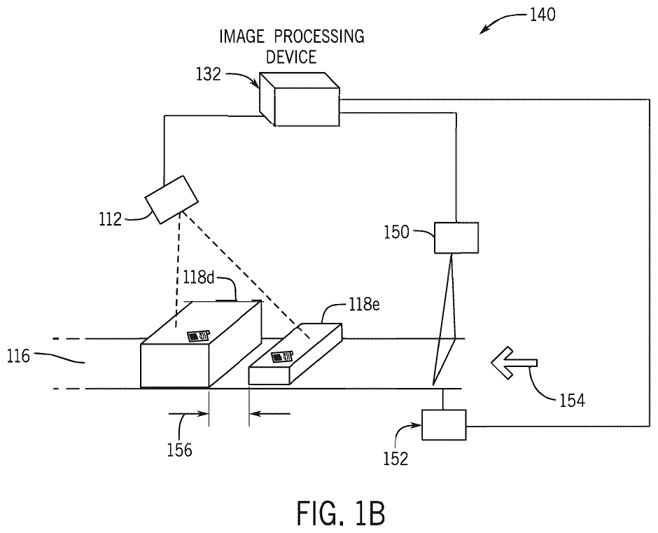

FIG. 1A illustrates an example of a system 100 for capturing multiple images of each side of an object in accordance with an embodiment of the technology. In some embodiments, the system 100 may be configured to evaluate symbols (e.g., barcodes, two-dimensional (“2D”) codes, fiducials, hazmat, and other labels) on objects (e.g., objects 118a, 118b) moving through a tunnel 102, such as a symbol 120 on object 118a, including assigning symbols to objects (e.g., the objects 118a, 118b). In some embodiments, the symbol 120 is a flat barcode on a top surface of the object 118a, and the objects 118a and 118b are roughly cuboid boxes. Additionally, or alternatively, in some embodiments, any suitable geometries are possible for an object to be imaged, and any variety of symbols and symbol locations may be imaged and evaluated, including non-direct part mark (“DPM”) symbols and DPM symbols located on a top or any other side of an object. Alternatively, or in addition, in some embodiments, a non-symbol recognition approach may be implemented. As one example, some embodiments can include a vision-based recognition of non-symbol based features, such as, e.g., one or more edges of an object.

As illustrated in FIG. 1A, the objects 118a and 118b are disposed on a conveyor 116. The conveyor 116 is configured to move the objects 118a and 118b in a direction of travel (e.g., horizontally from left-to-right) through the tunnel 102 at a relatively predictable and continuous rate, or at a variable rate measured by a device, such as, e.g., a motion measurement device (e.g., an encoder). Additionally, or alternatively, the objects 118a and 118b may move through the tunnel 102 in other ways (e.g., with non-linear movement). Although the embodiments described herein are described with respect to a conveyor type transport system, it should be understood that the embodiments described herein may be implemented with other types of transport systems.

In some embodiments, the system 100 may include one or more imaging devices 112 and an image processing device 132. As one example, the system 100 may include multiple imaging devices 112 in a tunnel arrangement (e.g., implementing a portion of the tunnel 102), representatively shown via the imaging devices 112a, 112b, and 112c, each with a field-of-view (“FOV”), representatively shown via FOV 114a, 114b, 114c, that includes part of the conveyor 116.

In some configurations, the system 100 may include additional or fewer imaging devices 112 than illustrated in FIG. 1A in various configurations. As one example, the system 100 may include a single imaging device, such as (a) the imaging device 112a, (b) the imaging device 112b, or (c) the imaging device 112c. As another example, the system 100 may include two imaging devices, such as (a) the imaging device 112a and the imaging device 112b, (b) the imaging device 112a and the imaging device 112c, or (c) the imaging device 112b and the imaging device 112c. In yet another example, the system 100 may include additional imaging devices than illustrated in FIG. 1A. Accordingly, the system 100 may include any number of imaging devices, including a single imaging device.

In some embodiments, each imaging device 112 may be positioned at an angle relative to the conveyor top or side (e.g., at an angle relative to a normal direction of symbols on the sides of the objects 118a and 118b or relative to the direction of travel), resulting in an angled FOV. Similarly, some of the FOVs may overlap with other FOVs (e.g., the FOV 114a and the FOV 114b). In such embodiments, the system 100 may be configured to capture one or more images of multiple sides of the objects 118a and/or 118b as the objects 118A and/or 118b are moved by the conveyor 116. In some embodiments, the captured images may be used to identify symbols on each object (e.g., a symbol 120) and/or assign symbols to each object, which may be subsequently decoded or analyzed (as appropriate). In some embodiments, a gap in the conveyor 116 (not shown) may facilitate imaging of a bottom side of an object (e.g., as described in U.S. Patent Application Publication No. 2019/0333259, filed on Apr. 25, 2018, which is hereby incorporated by reference herein in its entirety) using an imaging device or array of imaging devices disposed below the conveyor 116 (not illustrated). In some embodiments, the captured images from a bottom side of the object may also be used to identify symbols on the object and/or assign symbols to each object, which may be subsequently decoded (as appropriate). Note that although two arrays of three imaging devices 112 are shown imaging a top of objects 118a and 118b, and four arrays of two imaging devices 112 are shown imaging sides of objects 118a and 118b, this is merely an example, and any suitable number of imaging devices 112 may be used to capture images of one or more various sides of objects (e.g., including a single imaging device used to capture image(s) of a single side of an object). As one example, each array may include four or more imaging devices 112. As another example, the system 100 may include a single imaging device (as opposed to arrays including multiple imaging devices).

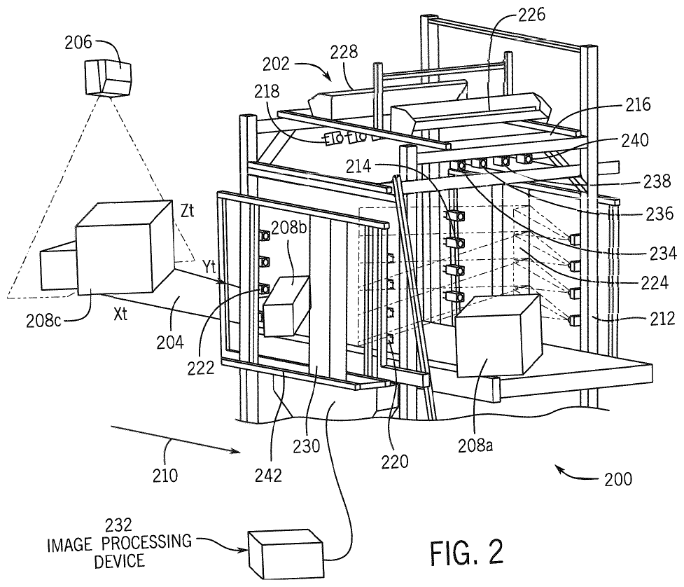

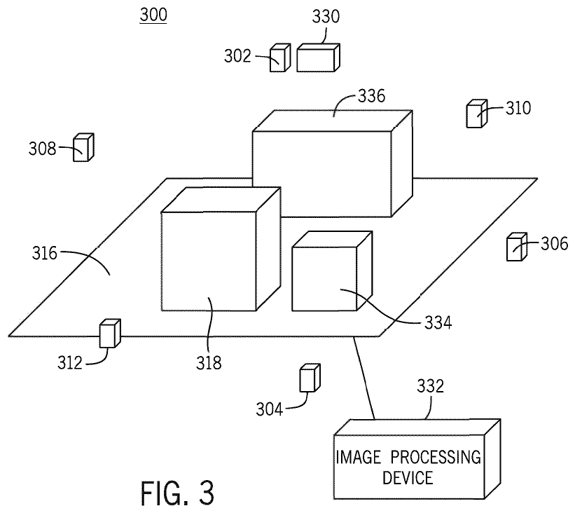

Additionally, although the imaging device(s) 112 are generally shown imaging the objects 118a and 118b without mirrors to redirect a FOV, this is merely one example, and one or more fixed and/or steerable mirrors may be used to redirect a FOV of one or more of the imaging device(s) 112 as described below with respect to FIGS. 2 and 3, which may facilitate a reduced vertical or lateral distance between the imaging device(s) 112 and the objects 118a, 118b in the tunnel 102. For example, the imaging device 112a may be disposed with an optical axis parallel to the conveyor 116, and one or more mirrors may be disposed above the tunnel 102 to redirect a FOV from the imaging device 112a toward a front and top of the objects 118a, 118b in the tunnel 102.

In some embodiments, the imaging device(s) 112 may be implemented using any suitable type of imaging device. As one example, the imaging device 112 may be implemented using a 2D imaging device (e.g., 2D camera), such as an area scan camera and/or line scan camera. In some embodiments, the imaging device 112 may be an integrated system that includes a lens assembly and an imager, such as a CCD or CMOS sensor. In some embodiments, the imaging device 112 may include one or more image sensors, at least one lens arrangement, and at least one control device (e.g., an electronic processor device) configured to execute computational operations relative to the image sensor(s). Each of the imaging devices 112a, 112b, or 112c may selectively acquire image data from different FOVs, regions of interest (“ROIs”), or a combination thereof. In some embodiments, the system 100 may be utilized to acquire multiple images of each side of an object where one or more images may include more than one object. Alternatively, in some embodiments, the system 100 may utilize a single imaging device to acquire multiple images of at least one side of an object where one or more images may include more than one object. The multiple images of each side may be used to assign a symbol in an image to an object in the image. The object 118a, 118b, 118c may be associated with one or more symbols, such as a barcode, a QR code, etc. In some embodiments, the system 100 may be configured to facilitate imaging of the bottom side of an object supported by the conveyor 116 (e.g., the side of the object 118a, 118b, 118c resting on the conveyor 116). As one example, the conveyor 116 may be implemented with a gap.

In some embodiments, a gap 122 is provided between objects 118a, 118b. In different implementations, gaps between objects may range in size. In some implementations, gaps between objects may be substantially the same between all sets of objects in a system, or may exhibit a fixed minimum size for all sets of objects in a system. In some embodiments, smaller gap sizes may be used to maximize system throughput.

In some embodiments, the system 100 may include a dimensioning system (not shown), sometime referred to herein as a dimensioner. The dimensioner may measure dimensions of objects moving toward the tunnel 102 on the conveyor 116. The dimensions may be used (e.g., by the image processing device 132) in a process to assign a symbol to an object in an image captured as one or more objects move through tunnel 102. Additionally, the system 100 may include devices (e.g., a motion measurement device, such as, e.g., an encoder, not shown) to track the physical movement of objects (e.g., objects 118a, 118b, 118c) moving through the tunnel 102 on the conveyor 116.

FIG. 1B shows an example of a system 140 for capturing multiple images of each side of an object 118d, 118e in accordance with an embodiment of the technology. FIG. 1B shows a simplified diagram of the system 140 to illustrate an example arrangement of a dimensioner and a motion measurement device (e.g., an encoder) with respect to a tunnel. As mentioned above, the system 140 may include a dimensioner 150 and a motion measurement device 152. In the illustrated example, the conveyor 116 is configured to move the objects 118d, 118e along the direction indicated by arrow 154 past the dimensioner 150 before the objects 118d, 118e are imaged by the imaging device(s) 112. In the illustrated example, the system 140 includes a single imaging device 112. However, in some configurations, the system 140 may include additional imaging devices 112 than illustrated in FIG. 1B.

In the illustrated embodiment, a gap 156 is provided between objects 118d and 118e. The image processing device 132 may be in communication with the imaging device 112, the dimensioner 150, and the motion measurement device 152. The dimensioner 150 may be configured to determine dimensions and/or a location of an object supported by a support structure (e.g., the object 118d or 118e) at a certain point in time. As one example, the dimensioner 150 may be configured to determine a distance from the dimensioner 150 to a top surface of the object 118d, 118e, and may be configured to determine a size and/or orientation of a surface facing the dimensioner 150.

In some embodiments, the dimensioner 150 may be implemented using various technologies. As one example, the dimensioner 150 may be implemented using a 3D camera (e.g., a structured light 3D camera, a continuous time of flight 3D camera, etc.). As another example, the dimensioner 150 may be implemented using a laser scanning system (e.g., a LiDAR system). In a particular example, the dimensioner 150 may be implemented using a 3D-A1000 system available from Cognex Corporation. In some embodiments, the dimensioning system or the dimensioner 150 (e.g., a time-of-flight sensor or computed from stereo) may be implemented in a single device or enclosure with the imaging device 112 (e.g., a 2D camera) and, in some embodiments, an electronic processor (e.g., that may be utilized as the image processing device 132) may also be implemented in the device with the dimensioner 150 and the imaging device 112. As used herein, unless otherwise specified, “electronic processor” is intended to encompass a wide range of processor devices, including with distributed (e.g., parallel or spatially separated) processing capabilities.

In some embodiments, the dimensioner 150 may determine 3D coordinates of each corner of the object 118d, 118e in a coordinate space defined with reference to one or more portions of the system 140. As one example, the dimensioner 150 may determine 3D coordinates of each of eight corners of an object 118d, 118e that is at least roughly cuboid in shape within a Cartesian coordinate space defined with an origin at the dimensioner 150. As another example, the dimensioner 150 may determine 3D coordinates of each of eight corners of an object 118d, 118e that is at least roughly cuboid in shape within a Cartesian coordinate space defined with respect to the conveyor 116 (e.g., with an origin that originates at a center of the conveyor 116).

In some embodiments, the motion measurement device 152 may be linked to the conveyor 116 and the imaging device 112 to provide electronic signals to the imaging device 112 and/or the image processing device 132 that indicate the amount of travel of the conveyor 116, and the objects 118d, 118e supported thereon, over a known amount of time. This may be useful, for example, in applications where the system 140 includes multiple imaging devices 112, in order to coordinate capture of images of particular objects (e.g., the objects 118d, 118e), based on calculated locations of the object 118d, 118e relative to a field of view of a relevant imaging device (e.g., the imaging device(s) 112). In some embodiments, the motion measurement device 152 may be configured to generate a pulse count (e.g., an encoder pulse count) that may be used to identify the position of the conveyor 116 along the direction of travel (e.g., the direction of the arrow 154). As one example, the motion measurement device 152 may provide a pulse count (e.g., an encoder pulse count) to the image processing device 132 for identifying and tracking the positions of objects (e.g., the objects 118d, 118e) on the conveyor 116. In some embodiments, the motion measurement device 152 may increment a pulse count (e.g., an encoder pulse count) each time the conveyor 116 moves a predetermined distance (as a pulse count distance) in the direction of the arrow 154. In some embodiments, a position of an object 118d, 118e may be determined based on an initial position, the change in the pulse count, and the pulse count distance.

In some embodiments, the image processing device 132 (or a control device) may coordinate operations of various components of the system 100, 140. For example, the image processing device 132 may control a dimensioner (e.g., the dimensioner 150 illustrated in FIG. 1B) to acquire dimensions of an object positioned on the conveyor 116 and may cause the imaging devices 112 to capture images of each side of the object positioned on the conveyor 116. In some embodiments, the image processing device 132 may control detailed operations of each imaging device 112, for example, by providing trigger signals to cause the imaging device 112 to capture images at particular times, etc. Alternatively, or in addition, in some embodiments, the image processing device 132 may configure other devices to acquire images with different parameters (as opposed to typical production operation parameters). Alternatively, in some embodiments, another device (e.g., an electronic processor included in one or more of the imaging devices 112, a separate controller device, etc.) may control detailed operations of each of the one or more imaging devices 112. As one example, the image processing device 132 (and/or any other suitable device) may provide a trigger signal to each imaging device 112 and/or dimensioner (e.g., the dimensioner 150 illustrated in FIG. 1B), and an electronic processor of each imaging device 112 may be configured to implement a predesignated image acquisition sequence that spans a predetermined region of interest in response to the trigger.

Note that the system 100, 140 may include one or more light sources (not shown) to illuminate surfaces of an object 118. Operation of such light sources may be coordinated by a central device (e.g., the image processing device 132), and/or control may be decentralized (e.g., an imaging device 112 may control operation of one or more light sources, an electronic processor associated with one or more light sources may control operation of the light sources, etc.).

As one example, in some embodiments, the system 100, 140 may be configured to concurrently (e.g., at the same time or over a common time interval) acquire images of multiple sides of an object 118, including as part of a single trigger event. As one example, each imaging device 112 may be configured to acquire a respective set of one or more images over a common time interval. Additionally, or alternatively, in some embodiments, the imaging devices 112 may be configured to acquire the images based on a single trigger event. As one example, based on a sensor (e.g., a contact sensor, a presence sensor, an imaging device 112, etc.) determining that the object (e.g., the object(s) 118a-118e) has passed into the FOV of the imaging device(s) 112, the imaging device(s) 112 may concurrently acquire images of the respective sides of the object (e.g., the object(s) 118a-118e).

As mentioned above, one or more fixed and/or steerable mirrors may be used to redirect a FOV of one or more of the imaging devices 112, which may facilitate a reduced vertical or lateral distance between the one or more imaging devices 112 and the object(s) 118 in the tunnel 102. FIG. 2 illustrates another example of a system 200 for capturing multiple images of each side of an object 208a, 208b in accordance with an embodiment of the technology. The system 200 includes multiple banks of imaging devices 212, 214, 216, 218, 220, 222 and multiple mirrors 224, 226, 228, 230 in a tunnel arrangement 202.

In some configurations, the tunnel arrangement 202 may include additional, different, or fewer components than illustrated in the example of FIG. 2 in various configurations or arrangements. For instance, in some examples, the system 200 includes a single bank of imaging devices, a single imaging device, a single mirror, etc. As another example, in some instances, each bank of imaging devices may include additional or fewer imaging devices than illustrated in the example arrangement of FIG. 2.

As one example, the banks of imaging devices illustrated in FIG. 2 include a left trail bank 212, a left lead bank 214, a top trail bank 216, a top lead bank 218, a right trail bank 220, and a right lead bank 222. In the illustrated embodiment, each bank 212, 214, 216, 218, 220, 222 includes four imaging devices that are configured to capture images of one or more sides of an object (e.g., the object 208a) and various FOVs of the one or more sides of the object 208a, 208b. As one example, the top trail bank 216 and the mirror 228 may be configured to capture images of the top and back surfaces of the object 208a using the imaging devices 234, 236, 238, and 240. In the illustrated embodiment, the banks of imaging devices 212, 214, 216, 218, 220, 222 and the mirrors 224, 226, 228, 230 may be mechanically coupled to a support structure 242 above a conveyor 204. Note that although the illustrated mounting positions of the banks imaging devices 212, 214, 216, 218, 220, 222 relative to one another may be advantageous, in some embodiments, imaging devices for imaging different sides of an object may be reoriented relative to the illustrated positions in FIG. 2 (e.g., imaging devices may be offset, imaging devices may be placed at the corners, rather than the sides, etc.). Similarly, while there may be advantages associated with using four imaging devices per bank that are each configured to acquire image data from one or more sides of an object, in some embodiments, a different number or arrangement of imaging devices (including a single imaging device), a different arrangement of mirror (e.g., using steerable mirrors, using additional fixed mirrors, etc.) may be used to configure a particular imaging device to acquire images of multiple sides of an object. In some embodiments, an imaging device may be dedicated to acquiring images of multiple sides of an object including with overlapping acquisition areas relative to other imaging devices included in the same system.

In some embodiments, the system 200 also includes a dimensioner 206 and an image processing device 232. As discussed above, multiple objects 208a, 208b and 208c may be supported in the conveyor 204 and travel through the tunnel arrangement 202 along a direction indicated by arrow 210. In some embodiments, each bank of imaging devices 212, 214, 216, 218, 220, 222 (and each imaging device in a bank) may generate a set of images depicting a FOV or various FOVs of a particular side or sides of an object supported by the conveyor 204 (e.g., the object 208a).

Note that although FIGS. 1A-1B and 2 depict a dynamic support structure (e.g., the conveyor 116, the conveyor 204) that is moveable, in some embodiments, a stationary support structure may be used to support objects to be imaged by one or more imaging devices. FIG. 3 shows another example system for capturing multiple images of each side of an object in accordance with an embodiment of the technology. In some embodiments, the system 300 may include multiple imaging devices 302, 304, 306, 308, 310, and 312, which may each include one or more image sensors, at least one lens arrangement, and at least one control device (e.g., a processor device) configured to execute computational operations relative to the image sensor. In some embodiments, the system 300 may include additional, different, or fewer components than illustrated in FIG. 3. For example, in some configurations, the system 300 may include a single imaging device, such as, e.g., the imaging device 302, the imaging device 304, the imaging device 306, the imaging device 308, the imaging device 310, or the imaging device 312. Accordingly, the system 300 may any number or combination of imaging devices, such as, e.g., three imaging devices, four imaging devices, etc.

In some embodiments, imaging devices 302, 304, 306, 308, 310, and/or 312 may include and/or be associated with a steerable mirror (e.g., as described in U.S. application Ser. No. 17/071,636, filed on Oct. 13, 2020, which is hereby incorporated by reference herein in its entirety). Each of the imaging devices 302, 304, 306, 308, 310, and/or 312 may selectively acquire image data from different fields of view (FOVs), corresponding to different orientations of the associated steerable mirror(s). In some embodiments, the system 300 may be utilized to acquire multiple images of each side of an object.

In some embodiments, the system 300 may be used to acquire images of multiple objects presented for image acquisition. As one example, the system 300 may include a support structure that supports each of the imaging devices 302, 304, 306, 308, 310, 312 and a platform 316 configured to support one or more objects 318, 334, 336 to be imaged (note that each object 318, 334, 336 may be associated with one or more symbols, such as a barcode, a QR code, etc.). As one example, a transport system (not shown), including one or more robot arms (e.g., a robot bin picker), may be used to position multiple objects (e.g., in a bin or other container) on the platform 316. In some embodiments, the support structure may be configured as a caged support structure. However, this is merely an example, and the support structure may be implemented in various configurations. In some embodiments, the support platform 316 may be configured to facilitate imaging of the bottom side of one or more objects supported by the support platform 316 (e.g., the side of an object 318, 334, or 336 resting on the platform 316). As one example, the support platform 316 may be implemented using a transparent platform, a mesh or grid platform, an open center platform, or any other suitable configuration. Other than the presence of the support platform 316, acquisition of images of the bottom side can be substantially similar to acquisition of other sides of the object.

In some embodiments, the imaging devices 302, 304, 306, 308, 310, and/or 312 may be oriented such that a FOV of the imaging device may be used to acquire images of a particular side of an object resting on the support platform 316, such that each side of an object (e.g., the object 318) placed on and supported by the support platform 316 may be imaged by the imaging devices 302, 304, 306, 308, 310, and/or 312. As one example, the imaging device 302 may be mechanically coupled to the support structure above the support platform 316, and may be oriented toward an upper surface of support platform 316, the imaging device 304 may be mechanically coupled to the support structure below the support platform 316, and the imaging devices 306, 308, 310, and/or 312 may each be mechanically coupled to a side of the support structure, such that a FOV of each of the imaging devices 306, 308, 310, and/or 312 faces a lateral side of the support platform 316.

In some embodiments, each imaging device may be configured with an optical axis that is generally parallel with another imaging device, and perpendicular to other imaging devices (e.g., when the steerable mirror is in a neutral position). As one example, the imaging devices 302 and 304 may be configured to face each other (e.g., such that the imaging devices 302 and 304 have substantially parallel optical axes), and the other imaging devices may be configured to have optical axis that are orthogonal to the optical axis of the imaging devices 302 and 304.

Note that although the illustrated mounting positions of the imaging devices 302, 304, 306, 308, 310, and 312 relative to one another may be advantageous, in some embodiments, imaging devices for imaging different sides of an object may be reoriented relative the illustrated positions of FIG. 3 (e.g., imaging device may be offset, imaging devices may be placed at the corners, rather than the sides, etc.). Similarly, while there may be advantages (e.g., increased acquisition speed) associated with using six imaging devices that is each configured to acquire imaging data from a respective side of an object (e.g., the six side of the object 118), in some embodiments, a different number or arrangement of imaging devices (including a single imaging device), a different arrangement of mirrors (e.g., using fixed mirrors, using additional moveable mirrors, etc.) may be used to configure a particular imaging device to acquire images of multiple sides of an object. As one example, fixed mirrors disposed such that the imaging devices 306 and 310 may capture images of a far side of the object 318 and may be used in lieu of the imaging devices 308 and 312. In some embodiments, the system 300 may be configured to image each of the multiple objects 318, 334, 336 on the platform 316.

In some embodiments, the system 300 may include a dimensioner 330. As described above with respect to FIGS. 1A, 1B and 2, the dimensioner 330 may be configured to determine dimensions and/or a location of an object supported by the support platform 316 (e.g., the object 318, 334, or 336). As mentioned above, in some embodiments, the dimensioner 330 may determine 3D coordinates of each corner of the object in a coordinate space defined with reference to one or more portions of the system 300. As one example, the dimensioner 330 may determine 3D coordinates of each of eight corners of an object that is at least roughly cuboid in shape within a Cartesian coordinate space defined with an origin at the dimensioner 330. As another example, the dimensioner 330 may determine 3D coordinates of each of eight corners of an object that is at least roughly cuboid in shape within a Cartesian coordinate space defined with respect to the support platform 316 (e.g., with an origin that originates at a center of the support platform 316).

In some embodiments, an image processing device 332 may coordinate operations of the imaging device(s) 302, 304, 306, 308, 310, and/or 312 and/or may perform image processing tasks as described above in connection with the image processing device 132 of FIG. 1A.

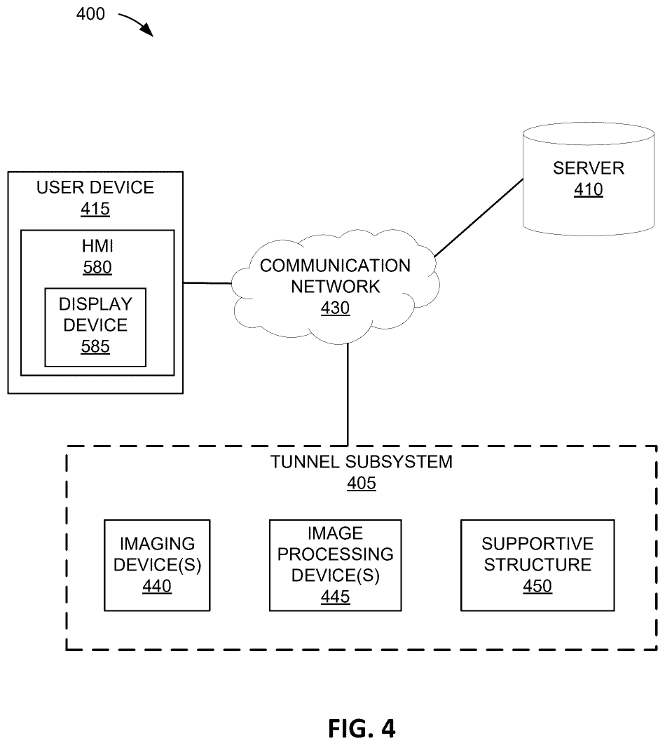

FIG. 4 schematically illustrates a system 400 for commissioning a machine vision system of a tunnel according to some embodiments. In the illustrated example, the system 400 includes a tunnel subsystem 405, a server 410, and a user device 415. In some embodiments, the system 400 includes fewer, additional, or different components in different configurations than illustrated in FIG. 4. As one example, the system 400 may include multiple tunnel subsystems 405, multiple servers 410, multiple user devices 415, or a combination thereof. As another example, one or more components of the system 400 may be combined into a single device, such as, e.g., the server 410 and a database.

The tunnel subsystem 405, the server 410, and the user device 415 communicate over one or more wired or wireless communication networks 430. Portions of the communication networks 430 may be implemented using a wide area network, such as the Internet, a local area network, such as a Bluetooth™ network or Wi-Fi, and combinations or derivatives thereof. Alternatively, or in addition, in some embodiments, components of the system 400 communicate directly as compared to through the communication network 430. Also, in some embodiments, the components of the system 400 communicate through one or more intermediary devices not illustrated in FIG. 4.

As illustrated in FIG. 4, the tunnel subsystem 405 may include one or more imaging devices 440 (referred to herein collectively as “the imaging devices 440” and individually as “the imaging device 440”), an image processing device 445, and a support structure 450. The imaging devices 440, the image processing device 445, and the support structure 450 may communicate wirelessly, over one or more communication lines or buses, or a combination thereof. In some embodiments, the tunnel subsystem 405 includes fewer, additional, or different components in different configurations than illustrated in FIG. 4.

For example, in some embodiments, the tunnel subsystem 405 may include the example systems (or component(s) thereof) as illustrated in FIGS. 1A-1B, 2, and 3 (e.g., the systems 100, 140, 200, and/or 300). As one example, the tunnel subsystem 405 may include a dimensioning system, including, e.g., one or more of the dimensioners 150, 206, and/or 330, as described in greater detail above. As another example, the tunnel subsystem 405 may include multiple image processing devices 445, multiple support structures 450, and the like. As yet another example, in some configurations, the tunnel subsystem 405 may include a single imaging device 440. Alternatively, in other configurations, the tunnel subsystem 405 may include multiple imaging devices 440. As yet another example, one or more components of the tunnel subsystem 405 may be combined into a single device. For example, as noted above, in some embodiments, each of the one or more imaging devices 440 may be associated with an associated image processing device (e.g., the image processing device 445), such that, in some embodiments, the tunnel subsystem 405 may include multiple image processing devices 445. In such embodiments, the imaging device 440 and the associated image processing device 440 may be included within the same housing or combined within a single device.

Accordingly, the tunnel subsystem 405 of FIG. 4 may include functionality and/or components similar to those described above with respect to FIGS. 1A-1B, 2, and 3. For example, the imaging device 440 may be similar to, e.g., the imaging device(s) 112, 234, 236, 238, and/or 240 as described above in greater detail, the image processing device 445 may be similar to, e.g., the image processing device(s) 132, 232, and/or 332 as described above in greater detail, the support structure 450 may be similar to, e.g., the conveyor 116, and/or 204 and/or the platform 316 as described above in greater detail.

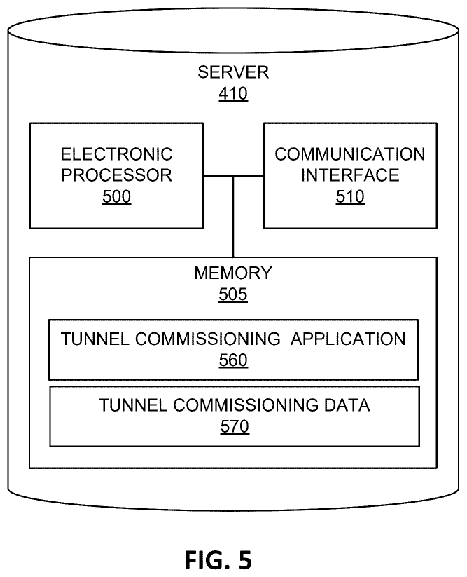

The server 410 is a computing device, such as a server, a database, or the like. As illustrated in FIG. 5, the server 410 includes an electronic processor 500, a memory 505, and a communication interface 510. The electronic processor 500, the memory 505, and the communication interface 510 communicate wirelessly, over one or more communication lines or buses, or a combination thereof. The server 410 may include additional components than those illustrated in FIG. 5 in various configurations. For example, the server 410 may also communicate with or include one or more human machine interfaces, such as a keyboard, keypad, mouse, joystick, touchscreen, display device, printer, speaker, and the like, that receive input from a user, provide output to a user, or a combination thereof. The server 410 may also perform additional functionality other than the functionality described herein. Also, the functionality described herein as being performed by the server 410 may be distributed among multiple servers or devices (e.g., as part of a cloud service or cloud-computing environment), combined with another component of the system 400 (e.g., combined with the user device 415, one or more components of the tunnel subsystem 405, or the like), or a combination thereof.

The communication interface 510 may include a transceiver that communicates with the tunnel subsystem 405, the user device 415, or a combination thereof over the communication network 430 and, optionally, one or more other communication networks or connections. The electronic processor 500 includes a microprocessor, an application-specific integrated circuit (“ASIC”), or another suitable electronic device for processing data, and the memory 505 includes a non-transitory, computer-readable storage medium. The electronic processor 500 may retrieve instructions and data from the memory 505 and execute the instructions.

For example, as illustrated in FIG. 5, the memory 505 may include a tunnel commissioning application 560 (referred to herein as “the application 560”). The application 560 is a software application executable by the electronic processor 500 in the example illustrated and as specifically discussed below, although a similarly purposed module can be implemented in other ways in other examples. As described in more detail below, the electronic processor 500 executes the application 560 to commission a tunnel, and, more specifically, commission a tunnel by identifying one or more imaging devices (e.g., the imaging device(s) 440) associated with a tunnel. In some instances, the electronic processor 500 executes the application 560 to automatically identify one or more imaging devices (e.g., the imaging device(s) 440), such as part of an auto-naming process. Alternatively, or in addition, in some configurations, the electronic processor 500 executes the application 560 to facilitate manual identification of one or more imaging devices (e.g., the imaging device(s) 440) based on user input, as part of a manual naming process.

In some embodiments, the application 560 commissions a tunnel using tunnel commissioning data 570. For example, in some embodiments, the application 560 receives a commissioning request associated with a tunnel and accesses the tunnel commissioning data 570 to identify one or more imaging devices (e.g., the imaging device(s) 440) associated with the tunnel. As illustrated in FIG. 5, the tunnel commissioning data 570 may be locally stored in the memory 505. However, alternatively, or in addition, in some embodiments, the tunnel commissioning data 570 may be remotely stored, such as, e.g., in a memory of the user device 415, a memory of a component of the tunnel subsystem 405, a remote database, or the like.

The tunnel commissioning data 570 may include specifications associated with a particular or specific tunnel. In some embodiments, the tunnel commissioning data 570 may include a tunnel configuration file, a tunnel configuration package, or a combination thereof. A tunnel configuration file may include, e.g., a device configuration file (e.g., an executable software file for configuring one or more devices associated with a corresponding tunnel). A tunnel configuration package can include an executable software file for configuring a tunnel. The tunnel configuration package may include application specific settings for the tunnel and/or component(s) thereof. The configuration package may include orientation data associated with an imaging device, a bank of imaging devices, etc. The tunnel configuration package may include a list of imaging devices 440 (where the list of imaging devices 440 may include a single imaging device or multiple imaging devices). The list of imaging devices 440 may include, e.g., for each imaging device 440 included in the list of imaging devices 440, an imaging device identifier, a standard internet protocol (“IP”) address, an application specific setting (including one or more scripts), coordinates (e.g., 3D coordinates) describing a position of the imaging device 440, and the like. In configurations including a bank of imaging devices, the list of imaging devices 440 may include a bank identifier of the imaging device 440, a bank location describing a location of the imaging device 440 within the bank, etc. The tunnel configuration file and the tunnel configuration package can be the same executable software file or can be separate executable software files. Alternatively, or in addition, the tunnel configuration file and the tunnel configuration package may be stored at different sources or the same source. As one example, the tunnel configuration file may be stored at a first storage device and the tunnel configuration package may be stored at a second storage device, different from the first storage device.

The user device 415 may be a computing device, such as a desktop computer, a laptop computer, a tablet computer, a terminal, a smart telephone, a smart television, a smart wearable, or another suitable computing device that interfaces with a user. Although not illustrated in FIG. 4, the user device 415 may include similar components as the server 410, such as electronic processor (e.g., a microprocessor, an ASIC, or another suitable electronic device), a memory (e.g., a non-transitory, computer-readable storage medium), a communication interface, such as a transceiver, for communicating over the communication network 430 and, optionally, one or more additional communication networks or connections. For example, to communicate with the server 410 (or another component of the system 400), the user device 415 may store a browser application or a dedicated software application executable by an electronic processor. The system 400 is described herein as providing tunnel commissioning service through the server 410. However, in other embodiments, the functionality described herein as being performed by the server 410 may be locally performed by the user device 415. For example, in some embodiments, the user device 415 may store the application 560, the tunnel commissioning data 570, or a combination thereof. As described in greater detail below, a user may use the user device 415 to commission a tunnel via, e.g., the application 560, the tunnel commissioning data 570, or a combination thereof.

In the illustrated example of FIG. 4, the user device 415 may also include a human-machine interface (“HMI”) 580 for interacting with a user. The HMI 580 may include one or more input devices, one or more output devices, or a combination thereof. Accordingly, in some embodiments, the HMI 580 allows a user to interact with (e.g., provide input to and receive output from) the user device 415. For example, the HMI 580 may include a keyboard, a cursor-control device (e.g., a mouse), a touch screen, a scroll ball, a mechanical button, a display device (e.g., a liquid crystal display (“LCD”)), a printer, a speaker, a microphone, or a combination thereof. As illustrated in FIG. 4, in some embodiments, the HMI 580 includes a display device 585. The display device 585 may be included in the same housing as the user device 415 or may communicate with the user device 415 over one or more wired or wireless connections. For example, in some embodiments, the display device 585 is a touchscreen included in a laptop computer or a tablet computer. In other embodiments, the display device 585 is a monitor, a television, or a projector coupled to a terminal, desktop computer, or the like via one or more cables.

As noted herein, the electronic processor 500 may execute the application 560 to commission a tunnel, and, more specifically, commission a tunnel by identifying one or more imaging devices (e.g., the imaging device(s) 440) associated with a tunnel. In some instances, the electronic processor 500 may execute the application 560 to automatically identify one or more imaging devices (e.g., the imaging device(s) 440), such as part of an auto-naming process. Alternatively, or in addition, in some configurations, the electronic processor 500 may execute the application 560 to facilitate manual identification of one or more imaging devices (e.g., the imaging device(s) 440) based on user input, as part of a manual naming process.

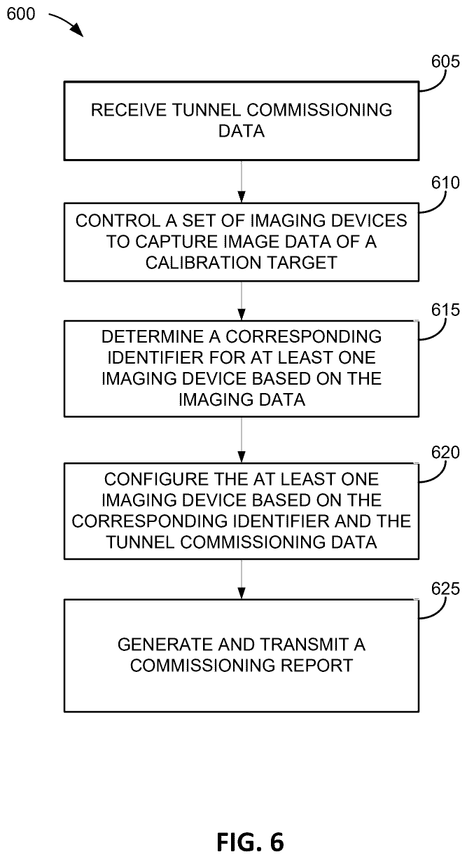

FIG. 6 is a flowchart illustrating a method 600 for commissioning a machine vision system for a tunnel using an auto-naming process for identifying one or more imaging devices associated with the tunnel according to some embodiments. The method 600 is described herein as being performed by the server 410 and, in particular, the application 560 as executed by the electronic processor 500. However, as noted above, the functionality described with respect to the method 600 may be performed by other devices, such as the user device 415, component(s) of the tunnel subsystem 405, or distributed among a plurality of devices, such as a plurality of servers included in a cloud service.

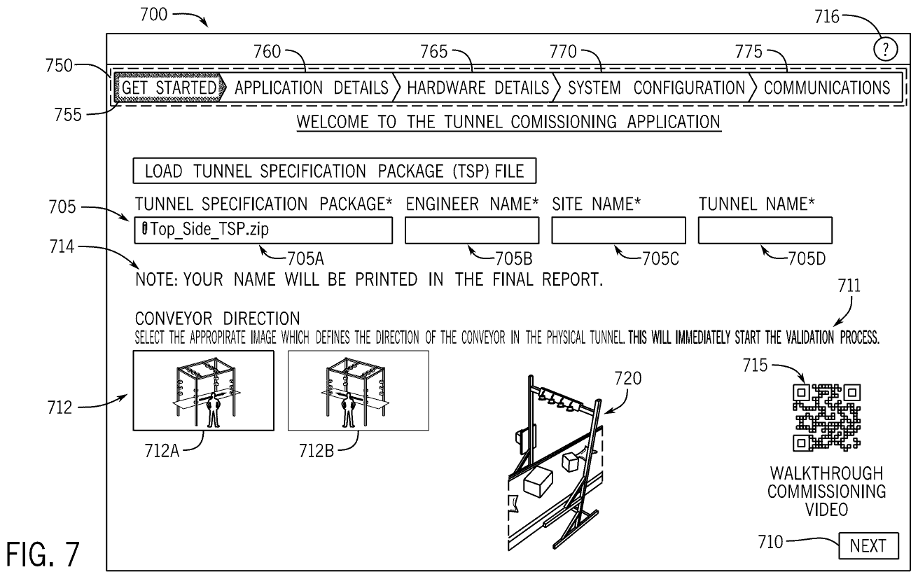

The method 600 is described herein with reference to FIGS. 7-22. FIGS. 7-22 are example screenshots of user interfaces for commissioning a machine vision system for a tunnel according to some embodiments. The user interface(s) of FIGS. 7-22 are non-limiting examples, and, in some embodiments, the user interface(s) may include additional, different, or fewer components and/or functionality than illustrated in FIGS. 7-22 and described herein. In some configurations, one or more of the user interfaces illustrated in FIGS. 7-22 may be combined, e.g., into a single user interface. Alternatively, or in addition, in some configurations, one or more of the user interfaces illustrated in FIGS. 7-22 may be separated into additional user interfaces.

As described in greater detail below, the electronic processor 500 may generate and provide one or more user interfaces associated with the commissioning of a tunnel and/or a machine vision system thereof. For example, the electronic processor 500 may provide or transmit (or otherwise output) the user interface(s) to a user such that the user may interact with the user interface(s), as described in greater detail below. In some embodiments, the electronic processor 500 may transmit the user interface(s) to a remote device, such as, e.g., the user device 415. In such embodiments, in response to receiving the user interface(s), the user device 415 may display the user interface(s) to a user of the user device 415 via the HMI 580 (e.g., the display device 585). Alternatively, or in addition, the electronic processor 500 may transmit user interface(s) to another component of the system 400, such as one or more components of the tunnel subsystem 405. As one example, the tunnel subsystem 405 may include an HMI (e.g., a display device) local to the tunnel subsystem 405, such as, e.g., a local control panel with a display device or touchscreen. According to this example, the electronic processor 500 may transmit the user interface(s) to the display device local to the tunnel subsystem 405. Alternatively, or in addition, the electronic processor 500 may transmit user interface(s) to an HMI associated with the server 410, such as, e.g., a display device of the server 410.

As illustrated in FIG. 6, the method 600 includes accessing, with the electronic processor 500, the tunnel commissioning data 570 (at block 605). As noted above, the tunnel commissioning data 570 may be locally stored in the memory 505. Accordingly, the electronic processor 500 may access the tunnel commissioning data 570 from the memory 505. Alternatively, or in addition, in some embodiments, the tunnel commissioning data 570 may be stored in a remote location, such as, e.g., in a memory of the user device 415, a component of the tunnel subsystem 405, a remote database or device, or the like. In such embodiments, the electronic processor 500 accesses the tunnel commissioning data 570 from a remote location.

In some embodiments, the electronic processor 500 accesses the tunnel commissioning data 570 in response to receiving a commissioning request (e.g., a request to commission a tunnel and/or a machine vision system of the tunnel). As one example, after a tunnel (or a machine vision system thereof) is physically assembled, a user may interact with the user device 415 to initiate a commissioning process for the physically assembled tunnel. The user may initiate the commissioning process by interacting with the application 560 via, e.g., the user device 415. As another example, in some embodiments, a user may interact with the user device 415 to initiate a re-commissioning process for a tunnel (or a machine vision system thereof) that is already in commission by, e.g., interacting with the application 560. In some examples, a user may initiate a re-commissioning process to confirm the tunnel is still operating as expected or desired.

In response to receiving the commissioning request, the electronic processor 500 may generate a user interface prompting a user for various commissioning parameters (i.e., any of various equipment, site, or tunnel-configuration parameters that specify a particular tunnel configuration). FIG. 7 illustrates an example commissioning details user interface 700 according to some embodiments. A user may interact with the commissioning details user interface 700 to provide commissioning parameters or information. Commissioning parameters may include, e.g., a tunnel identifier (i.e., a selection of a tunnel specification package or data), an operator identifier (i.e., an identifier of a user performing the commissioning processes), a site identifier (i.e., an identifier of a site where the commissioning processes is taking place to install or otherwise configure a particular tunnel). In some examples, a tunnel identifier can include identifiers relating to the structural components of the tunnel or machine vision system of the tunnel system being commissioned. In some configurations, commissioning parameters may include data associated with other system integrators or entities involved in the commissioning of a tunnel. For instance, in some examples, the commissioning parameters may include data identifying another integrator, an involvement of the other integrator, etc. Alternatively, or in addition, in some configurations, the commissioning parameters may include additional data or information associated with the tunnel being commissioned. For example, in some configurations, the commissioning parameters may include a conveyor direction.

In the illustrated example, the commissioning details user interface 700 may include a set of input mechanisms 705. A user may interact with the set of input mechanisms 705 by inputting information or selecting information related to commissioning the tunnel (or a machine vision system thereof). As one example, with reference to the illustrated example, a user may select a tunnel specification package using a first input mechanism 705A, input a user identifier using a second input mechanism 705B, input a site identifier using a third input mechanism 705C, and input a tunnel or station identifier using a fourth input mechanism 705D. In some configurations, the tunnel specification package may be an executable software file. Alternatively, or in addition, the tunnel specification package may be a dataset stored in and accessible from a remote device, such as, e.g., a database, a server, etc. As also illustrated in FIG. 7, the commissioning details user interface 700 may also include a “Next” button 710. A user may select the “Next” button 710 to indicate that the user is done inputting commissioning parameters.

In some configurations, the commissioning details user interface 700 includes a conveyor direction portion 711. The conveyor direction portion 711 may include GUI input elements for indicating a conveyor direction associated with the tunnel (or machine vision system) being commissioned. By interacting with one of the GUI input elements, the user may select a conveyor direction for the tunnel (or machine vision system thereof) being commissioned. For example, as illustrated in FIG. 7, the conveyor direction portion 711 includes a first GUI input element 712A and a second GUI input element 712B. The first GUI input element 712A may include a graphical representation of a virtual tunnel and a first conveyor direction. The second GUI input element 712B may include a graphical representation of the virtual tunnel and a second conveyor direction, where the second conveyor direction may be different from the first conveyor direction. In the example illustrated in FIG. 7, the first GUI input element 712A indicates a left-to-right conveyor direction and the second GUI input element 712 B indicates a right-to-left conveyor direction. In some configurations, the graphical representation may be based on the tunnel specification package. For instance, the virtual tunnel may resemble or represent the physical tunnel (or machine vision system thereof) being commissioned. Alternatively, or in addition, in some configurations, the conveyor direction options (e.g., the GUI input elements for indicating the conveyor direction) may be based on the tunnel specification package. For instance, the conveyor direction options may represent the specific conveyor direction options associated with the tunnel (or machine vision system thereof) being commissioned. As such, in some configurations, the conveyor direction portion 711 may include additional, different, or fewer GUI input elements for indicating the conveyor direction.

In some configurations, the commissioning details user interface 700 may include a banner showing a tunnel type at or proximate to a top portion of each user interface. The tunnel type or other tunnel-specific information may be provided from the tunnel specification package. In some configurations, the tunnel type or other tunnel-specific information may be repeated on one or more subsequently provided user interfaces. In some configurations, the commissioning details user interface 700 may include a notice 714. The notice 714 may provide notice to the user that the user is aware and takes responsibility for commissioning the tunnel (or the machine vision system thereof).

In some configurations, the commissioning details user interface 700 may provide instructions outlining the commissioning process. The instructions may include written text instructions, graphical instructions, video instructions, etc. In some configurations, the commissioning details user interface 700 may include written instructions. Alternatively, or in addition, in some configurations, the commissioning details user interface 700 may include a selectable link for accessing the instructions. In some examples, the commissioning details user interface 700 may include a hyperlink that, when selected by a user, navigates to or provides access to the instructions (e.g., a website including the instructions). As another example, as illustrated in FIG. 7, the commissioning details user interface 700 may include a barcode, such as a QR code 715, that a user may interact with in order to access the instructions (e.g., a walkthrough commissioning video). Inclusion of the QR code 715 may facilitate a user accessing the instructions in a situation where internet access is unavailable. Accordingly, in some configurations, the electronic processor 500 may generate a selectable link (e.g., a hyperlink, the QR code 715, etc.) that, when selected, provides access to instructions for commissioning the tunnel. In some configurations, the electronic processor 500 creates the QR code 715 for a video on how to run the application based on, e.g., a uniform resource locator (URL) provided by the tunnel specification package. Alternatively, or in addition, in some configurations, the commissioning details user interface 700 may include a “Hint” button 716 (as a GUI element). In response to a user interacting with the “Hint” button 716, the commissioning details user interface 700 may provide additional information or instructions to the user. For example, the additional information or instructions may include where to obtain the tunnel specification package, a point of contact when the tunnel specification package is not available to the user, etc.

In some configurations, the commissioning details user interface 700 may include a graphical representation of the tunnel (or machine visions system) being commissioned (represented in FIG. 7 by reference numeral 720). In some examples, the graphical representation 720 may include an image of the actual tunnel (or machine vision system) being commissioned. In some configurations, the electronic processor 500 may access the graphical representation 720 from the tunnel specification package, which may include an image of the actual tunnel (or machine vision system) being commissioned). The electronic processor 500 may include the graphical representation 720 from the tunnel specification package in the commissioning details user interface 700. Accordingly, in some configurations, the graphical representation 720 may be provided via the commissioning details user interface 700 after the tunnel specification package is accessible.

In some embodiments, the commissioning details user interface 700 includes a commissioning timeline 750. As illustrated in FIG. 7, the commissioning timeline 750 is a graphical representation of the steps included in the commissioning process according to some configurations. In the illustrated example, the commissioning timeline 750 may include a “Get Started” indicator 755, an “Application Details” indicator 760, a “Hardware Details” indicator 765, a “System Configuration” indicator 770, and a “Communications” indicator 775. The “Get Started” indicator 755 may be associated with a getting started or commissioning details stage of the commissioning process. The “Application Details” indicator 760 may be associated with an application details stage of the commissioning process. The “Hardware Details” indicator 765 may be associated with a hardware details or pre-commissioning checklist stage of the commissioning process. The “System Configuration” indicator 770 may be associated with a system configuration and naming stage of the commissioning process. The “Communications” indicator 775 may be associated with a communications stage of the commissioning process.

The commissioning timeline 750 is one non-limiting example of a commissioning timeline and, in some embodiments, the commissioning process (e.g., the commissioning timeline 750) includes additional, different, or fewer stages and/or indicators than illustrated in various configurations or orders. As one example, in some configurations, the technology disclosed herein implements an auto-naming process for one or more imaging devices of the tunnel being commissioned. Accordingly, in such configurations, the commissioning process (e.g., the commissioning timeline 750) may include the auto-naming stage (and a corresponding auto-naming indicator within the commissioning timeline 750). As another example, in some configurations, the commissioning process disclosed herein may include a methodology stage (and a corresponding methodology indicator 765 within the commissioning timeline 750), as described in greater detail herein. Additionally, it should be understood that the order of the stages of the commissioning process described herein is one non-limiting example order of the stages and that the order of the stages of the commissioning process may be different.

As a user progresses through each stage of the commissioning process (e.g., navigates from one user interface to the next), the electronic processor 500 may dynamically update the commissioning timeline 750 to indicate a current stage of the commissioning process. As one example, the electronic processor 500 may dynamically update the commissioning timeline 750 by altering a characteristic of one or more indicators (e.g., the “Get Started” indicator 755, the “Application Details” indicator 760, the “Hardware Details” indicator 765, the “System Configuration” indicator 770, and/or the “Communications” indicator 775). The electronic processor 500 may alter a characteristic of an indicator by, e.g., changing a color of the indicator, highlighting the indicator, changing a format property (e.g., a font style, a font size, a font property, such as bold, italics, capitols, etc., or the like), animating the indicator (e.g., flashing the indicator, etc.), placing a graphic around the indicator (e.g., displaying a box around the indicator), or the like. In some embodiments, the electronic processor 500 may dynamically update a single indicator. As one example, when the commissioning process advances to the application details stage, the electronic processor 500 may dynamically update the “Application Details” indicator 760, while the other indicators are not updated. Alternatively, in some embodiments, the electronic processor 500 may dynamically update more than one indicator. As one example, when the commissioning process advances from the hardware details stage to the system configuration stage, the electronic processor 500 may dynamically update the commissioning timeline 750 by greying-out the “Hardware Details” indicator 765 (thereby indicating that the hardware details stage is completed) and flashing the “System Configuration” indicator 770 (thereby indicating that the system configuration stage is the current commissioning stage).

In some configurations, after receiving the commissioning parameters (e.g., as a set of commissioning parameters), the electronic processor 500 may generate and provide application details. Application details may include, e.g., a tunnel type, a set of customer specifications, a set of application-specific requests, a set of tunnel capabilities, etc. Customer specifications may include, e.g., conveyor information, object information, etc. with respect to specifications of the customer. Conveyor information with respect to specifications of the customer may include, e.g., belt width, a minimum conveyor gap, a maximum line speed, etc. Object information with respect to specifications of the customer may include a maximum length, a maximum width, a maximum height, a minimum length, a minimum width, a minimum height, etc. Tunnel capabilities may include conveyor information, object information, etc. with respect to the capabilities of the tunnel. For instance, the conveyor information with respect to the capabilities of the tunnel may include, e.g., a belt width, a minimum gap, a maximum line speed, a working distance, a trigger distance, etc. The object information with respect to the capabilities of the tunnel may include, e.g., a maximum length, a maximum width, a maximum height, a minimum length, a minimum width, a minimum height, etc. Application-specific requests may include, e.g., a barcode location, etc. In some configurations, the application details may be based on content or information included within the tunnel specification package. Accordingly, the electronic processor 500 may generate and provide the application details by accessing the tunnel specification package. In some configurations, the electronic processor 500 may provide the application details as read-only data.

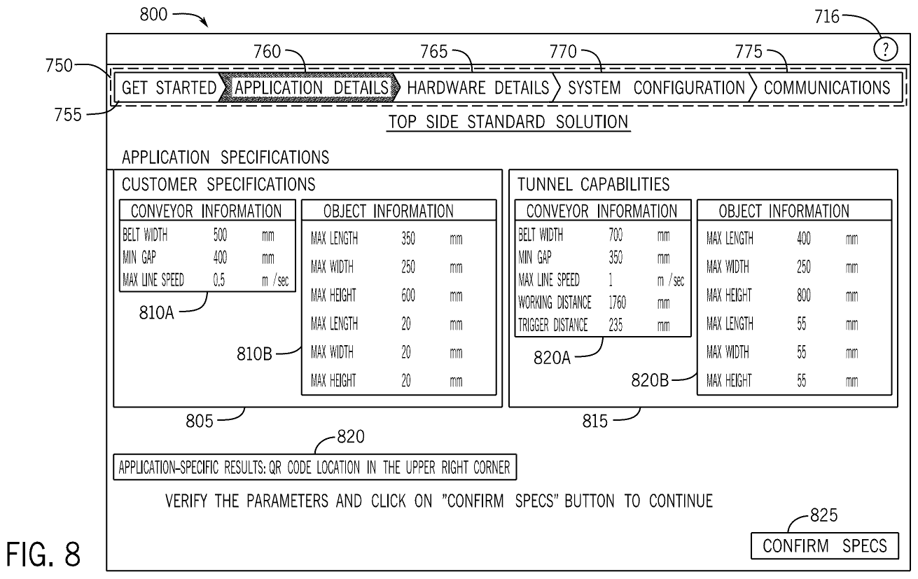

FIG. 8 illustrates an example application details user interface 800 according to some embodiments. As illustrated in the example, the application details user interface 800 may provide the application details, including, e.g., the tunnel type, the set of customer specifications, the set of application-specific requests, the set of tunnel capabilities, etc. As illustrated in FIG. 8, the application details user interface 800 may include a customer specifications portion 805. The customer specification portion 805 may include a set of customer specifications. In the illustrated example, the set of customer specifications may be provided in one or more tables, including, e.g., a conveyor information table 810A and an object information table 810B. As illustrated in FIG. 8, the application details user interface 800 may include a tunnel capabilities portion 815. The tunnel capabilities portion 815 may include a set of tunnel capabilities. In the illustrated example, the set of tunnel capabilities may be provided in one or more tables, including, e.g., a conveyor information table 820A and an object information table 820B. The application details user interface 800 may include an application-specific requests portion 820. The application-specific requests portion 820 may include a list of one or more application-specific requests.

A user may interact with the application details user interface 800 by viewing the application details provided via the application details user interface 800. In some configurations, the user may interact with the application details user interface 800 as part of a verification and confirmation process for the application details associated with the tunnel being commissioned. For instance, the user may be prompted to verify the information included in the application details user interface 800 and interact with a confirm button 825 (as a GUI control element). By a user interacting with the confirm button 825 (e.g., selecting or clicking the confirm button 825), the user may be verifying and confirming the information included in the application details user interface 800.

In some embodiments, after receiving verification and configuration of the application details (e.g., as a set of application details), the electronic processor 500 may generate and provide a pre-commissioning checklist. The pre-commissioning checklist may include a list of actions or tasks that a user may manually perform prior to commissioning the tunnel (e.g., as a set of pre-commissioning tasks). The set of pre-commissioning tasks may include, e.g., confirming a mechanical build of the tunnel, confirming a cable connection, confirming a network connection, confirming proper materials, confirming pre-work performance, confirming edge intelligence panel connections, and the like. In some embodiments, which pre-commissioning tasks are included in the pre-commissioning checklist is based on one or more of the commissioning parameters. Alternatively, or in addition, in some embodiments, the pre-commissioning checklist is based on (specific to) the tunnel (or machine vision system thereof) being commissioned.

Accordingly, in some embodiments, the electronic processor 500 generates the pre-commissioning checklist (and the pre-commissioning tasks included therein) based on the tunnel (or machine vision system thereof) being commissioned, the set of commissioning parameters, a customer specific setting, a site-specific setting, an application specific setting, or a combination thereof. As one example, when the tunnel being commissioned includes five different cable connections, the pre-commissioning checklist may include five different cable connection confirmation tasks specific to each of the five different cable connections. As another example, when the tunnel being commissioned includes an ethernet cable connected into a customer network switch, the pre-commissioning checklist may include a confirmation task specific to connecting an ethernet cable into a customer network switch. As yet another example, when the tunnel being commissioned includes a set of specific customer settings, the pre-commissioning checklist may include a confirmation task associated with each specific customer setting. As yet another example, when a first tunnel being commissioned at a first site includes two different cable connections and a second tunnel being commissioned at a second site include three different cable connections, the pre-commissioning checklist for the first tunnel can include two different cable connection confirmation tasks specific to each of the two different cable connection and the pre-commissioning checklist for the second tunnel can include three different cable connection confirmation tasks specific to each of the three different cable connections. Accordingly, in some embodiments, pre-commissioning checklists may differ from one customer to another customer, one application to another application, one site to another site, one tunnel to another tunnel, one workstation to another workstation, etc.

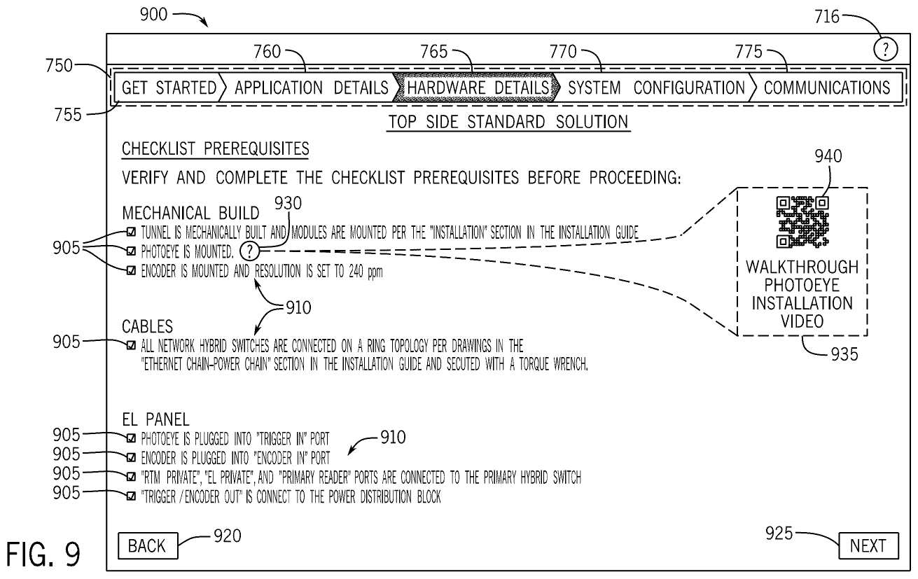

FIG. 9 illustrates an example pre-commissioning checklist user interface 900 associated with a hardware details stage of the commissioning process according to some embodiments. A user may interact with the pre-commissioning checklist user interface 900 via one or more input mechanisms (or GUI control elements) to confirm that each pre-commissioning task included in the set of pre-commissioning tasks has been completed. In the illustrated example, the input mechanisms are illustrated as checkboxes 905. As illustrated in FIG. 9, each checkbox 905 is associated with a corresponding pre-commissioning task 910 (e.g., a written description of a pre-commissioning task). As also illustrated in FIG. 9, the pre-commissioning checklist user interface 900 may include two navigation mechanisms (or GUI control elements), including a “Back” button 920 and a “Next” button 925. A user may interact with (e.g., select via a mouse click) the “Back” button 920 to navigate back to the application details user interface 800 of FIG. 8 (or another previous user interface). A user may interact with (e.g., select via a mouse click) the “Next” button 925 to indicate that the user has completed the set of pre-commissioning tasks. In some embodiments, the “Next” button 925 is not active (not selectable) until each input mechanism (e.g., the checkboxes 905) has been selected by a user (indicating that each pre-commissioning task has been completed by the user). Accordingly, in such embodiments, once of the checkboxes 905 is selected by the user, then the “Next” button 925 is activated such that a user may interact with the “Next” button 925. Similar to the application details user interface 800, in some embodiments, the pre-commissioning checklist user interface 900 includes the commissioning timeline 750. As illustrated in FIG. 9, the commissioning timeline 750 indicates that the user is at a hardware details stage of the commissioning process (represented by the altered appearance of the “Hardware Details” indicator 760 in comparison to FIG. 8).

In some configurations, the pre-commissioning checklist user interface 900 may include a help button (as a GUI element) associated with a pre-commissioning task. A user may interact with the help button in order to access additional information or instructions associated with the associated pre-commissioning task. For example, as illustrated in FIG. 9, a help button 930 may be associated with a “Photoeye is mounted” pre-commissioning task. In response to a user interacting with the help button 930, the electronic processor 500 may generate a help portion 935 for the “Photoeye is mounted” pre-commissioning task. In some instances, the electronic processor 500 may generate the help portion 935 within the pre-commissioning checklist user interface 900 (e.g., as part of or a component of the pre-commissioning checklist user interface 900). Alternatively, or in addition, the electronic processor 500 may generate the help portion 935 as a separate user interface (e.g., a pop-up window). The help portion 935 may include a selectable link for accessing the additional information or instructions. In some examples, the help portion 935 may include a hyperlink that, when selected by a user, navigates to or provides access to the instructions (e.g., a website including the instructions). As another example, as illustrated in FIG. 9, the help portion 935 may include a barcode, such as a QR code 940, that a user may interact with in order to access the instructions (e.g., a walkthrough photoeye installation video). Inclusion of the QR code 940 may facilitate a user accessing the instructions in a situation where internet access is unavailable. Accordingly, in some configurations, the electronic processor 500 may generate a selectable link (e.g., a hyperlink, the QR code 940, etc.) that, when selected, provides access to instructions for installing a photoeye. In some configurations, the electronic processor 500 creates the QR code 940 for a video on how to install the photoeye based on, e.g., a uniform resource locator (URL) provided by the tunnel specification package.

As noted herein, in some configurations, the commissioning process (e.g., the commissioning timeline 750) may include additional, different, or fewer stages. For instance, in some configurations, the technology disclosed herein may be implemented with a vision service without edge intelligence technology or functionality. In such configurations, the commissioning process (e.g., the commissioning timeline 750) may include a methodology stage.

Accordingly, in some embodiments, after receiving confirmation that each pre-commissioning task 910 is completed via user interaction with the pre-commissioning checklist user interface 900, the electronic processor 500 may prompt a user for methodology details. Methodology details or parameters may include, e.g., a commissioning methodology for identifying each imaging device 440 associated with the tunnel being commissioned. In some configurations, a commissioning methodology may relate to a process for naming one or more imaging devices of a machine vision system, such as, e.g., an auto-naming process, a manual naming process, etc. In some examples, methodology details can include a parameter associated with a calibration target, such as, e.g., a calibration target indicator (e.g., a model number of the calibration target), a material of the calibration target (e.g., metal, cardboard, etc.), one or more dimensions of the calibration target (e.g., as a set of dimensions), or the like.

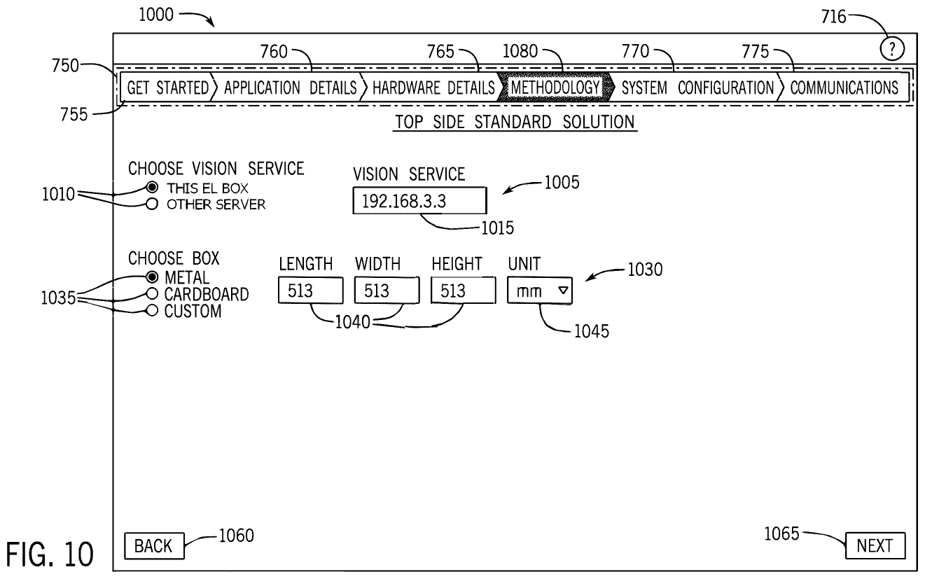

FIG. 10 illustrates an example methodology user interface 1000 according to some embodiments. A user may interact with the methodology user interface 1000 via one or more input mechanisms to provide a set of methodology details. In the illustrated example of FIG. 10, the methodology user interface 1000 includes a commissioning methodology portion 1005 associated with selecting one or more commissioning methodology related parameters. As illustrated, the commissioning methodology portion 1005 includes a first set of radio buttons 1010 for selecting which commissioning methodology to implement for commissioning the tunnel. Alternatively, or in addition, in some embodiments, the commissioning methodology portion 1005 may include a text field 1015. The text field 1015 may receive text input from a user. The text input may be used by the user to provide additional details related to the commissioning methodology (e.g., a specific version of a commissioning methodology), write a commissioning methodology not included in the first set of radio buttons 1010, or the like. The methodology user interface 1000 may also include a calibration target portion 1030 associated with selecting one or more calibration target parameters. In the illustrated example, the calibration target portion 1030 includes a second set of radio buttons 1035 for selecting a material parameter associated with the calibration target (e.g., “metal,” “cardboard,” and “custom”). The calibration target portion 1030 also includes a set of text fields 1040 for providing text input for the set of dimensions of the calibration target. The calibration target portion 1030 also includes a drop-down menu 1045 for selecting a unit of measurement associated with the set of dimensions.

As also illustrated in FIG. 10, the methodology user interface 1000 may include two navigation mechanisms, including a “Back” button 1060 and a “Next” button 1065. A user may interact with (e.g., select via a mouse click) the “Back” button 1060 to navigate to a previous user interface. A user may interact with (e.g., select via a mouse click) the “Next” button 1065 to navigate to the next user interface (e.g., the next stage of the commissioning process). In some embodiments, the “Next” button 1065 is not active (not selectable) information is provided in the commissioning methodology portion 1005, the calibration target portion 1030, or a combination thereof. In the illustrated example, the methodology user interface 1000 includes the commissioning timeline 750. As illustrated in FIG. 10, the commissioning timeline 750 may include a “Methodology” indicator 1080 indicating that the user is at a methodology stage of the commissioning process (represented by the altered appearance of the “Methodology” indicator 1080 in comparison to FIG. 10).

Returning to FIG. 6, the method 600 includes controlling, with the electronic processor 500, the set of imaging devices 440 to capture image data of a calibration target (at block 610). In some embodiments, the electronic processor 500 controls the imaging device(s) 440 to capture image data based on the set of application details, the set of commissioning parameters, the set of methodology details, the tunnel commissioning data, other data included in the tunnel specification package, or a combination thereof. Alternatively, or in addition, in some embodiments, the electronic processor 500 confirms that each of the one or more imaging devices 440 is discoverable (or discovered) prior to controlling the imaging device(s) 440 to capture the image data of the calibration target. As one example, the electronic processor 500 may wait until each of the one or more imaging devices 440 is discovered prior controlling the imaging device(s) 440 to capture the image data. Alternatively, or in addition, in some embodiments, the electronic processor 500 controls the imaging device(s) 440 to capture the image data in response to a user interacting with the “Next” button 925 of the pre-commissioning checklist user interface 900 of FIG. 9.

In response to capturing the image data of the calibration target (at block 610), the electronic processor 500 determines a corresponding identifier for at least one imaging device 440 based on the imaging data (at block 615). In some embodiments, the electronic processor 500 processes or analyzes the image data based on the set of application details, the set of commissioning parameters, the set of methodology details, the tunnel commissioning data, other data included in the tunnel specification package, or a combination thereof. Based on this analysis, the electronic processor 500 may determine an identifier status for each imaging device 440. The identifier status may indicate whether an imaging device 440 was associated with a corresponding identifier. Alternatively, or in addition, when the imaging device 440 was associated with a corresponding identifier, the identifier status may indicate the corresponding identifier. Alternatively, or in addition, when the imaging device 440 was not associated with a corresponding identifier, the identifier status may indicate that the imaging device 440 was not associated with a corresponding identifier.

In some embodiments, when the imaging device 440 was not associated with a corresponding identifier, the electronic processor 500 may determine a list of identifiers for the imaging device 440. The list of identifiers for the imaging device 440 may include a listing of identifiers that could be associated with the imaging device 440. Accordingly, in some embodiments, the list of identifiers includes a listing of suggested identifiers for the imaging device 440. In some embodiments, the electronic processor 500 determines the list of identifiers based on the tunnel commissioning data (e.g., data included in the tunnel specification package). As one example, where the tunnel commissioning data includes tunnel specifications indicating that the tunnel being commissioned includes five imaging devices, the electronic processor 500 may determine the list of identifiers to include the identifiers for the five imaging devices included in the tunnel specifications. Alternatively, or in addition, in some embodiments, the electronic processor 500 determines the list of identifiers for an imaging device 440 (e.g., a first imaging device) based on an identifier status of another imaging device 440 (e.g., a second imaging device). For example, the electronic processor 500 may determine the list of identifiers for an imaging device 440 based on which identifiers are already assigned to (or associated with) different imaging devices, where the list of identifiers for the imaging device 440 includes the remaining (or un-assigned) identifiers. Alternatively, or in addition, in some embodiments, the electronic processor 500 determines or updates a list of identifiers for an imaging device 440 based on a user interaction with a list of identifiers for another imaging device (e.g., a selection of an identifier for the other imaging device).

As one example, the tunnel commissioning data may include tunnel specification package data indicating that the tunnel being commissioned includes three known imaging devices each associated with a corresponding known identifier. Following this example, when the electronic processor 500 analyzes the image data, the electronic processor 500 may determine that a first known imaging device is associated with a first corresponding identifier. However, the electronic processor 500 may not be able to determine which corresponding identifier (either the second corresponding identifier or the third corresponding identifier) should be associated with the second known imaging device and the third known imaging device. Accordingly, the electronic processor 500 may determine that a list of identifiers for the second known imaging device includes a second corresponding identifier and a third corresponding identifier, where the second corresponding identifier and the third corresponding identifiers are the remaining (or un-assigned) identifiers of the tunnel specification package data. Similarly, the electronic processor 500 may determine that the list of identifiers for the third known imaging device includes the second corresponding identifier and the third corresponding identifier.

As noted above, in some embodiments, the electronic processor 500 updates a list of identifiers based on a user interaction with a list of identifiers for another imaging device. Following the above example, when a user interacts with the list of identifiers for the second known imaging device by selecting the second corresponding identifier for association with the second known imaging device, the electronic processor 500 may update the list of identifiers for the third known imaging device by removing the second corresponding identifier from the list of identifiers for the third known imaging device. Alternatively, or in addition, when the list of identifiers is updated such that the list of identifiers only includes one suggested corresponding identifier, the electronic processor 500 may automatically associate the remaining corresponding identifier with the last imaging device. Following the above example, when the second corresponding identifier is removed from the list of identifiers for the third known imaging device, the list of identifiers for the third known imaging device only includes the third corresponding identifier. Accordingly, in such situations, the electronic processor 500 may automatically determine that the third corresponding identifier is associated with the third known imaging device (as the third corresponding identifier is the only remaining identifier to be assigned).

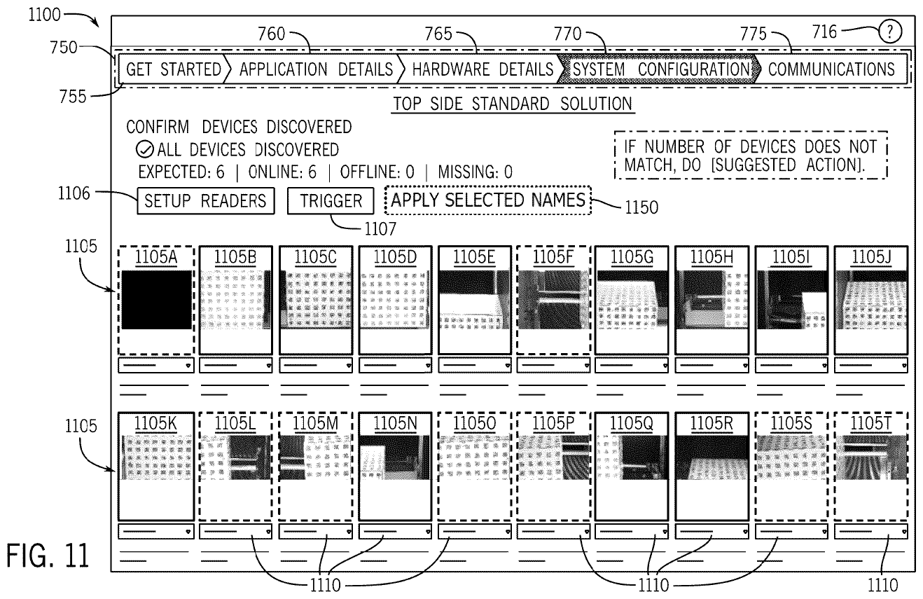

In some embodiments, the electronic processor 500 generates and provides a user interface including an identifier status for each imaging device 440. FIG. 11 illustrates an example auto-naming user interface 1100 according to some embodiments. In some configurations, the auto-naming user interface 1100 may be (or associated with) the system configuration stage of the commissioning process. Additionally, the auto-naming user interface 1100 of FIG. 11 is merely one example of an auto-naming user interface 1100. For instance, while the auto-naming user interface 1100 is described herein and depicted by the figures as being associated with an auto-naming process for a tunnel having multiple imaging devices, it should be understood that the auto-naming user interface 1100 may be implemented with an auto-naming process for a tunnel having a different number of imaging devices, including a single imaging device.

As illustrated in FIG. 11, the auto-naming user interface 1100 may include a set of preview images 1105. Each preview image 1105 may be associated with an imaging device 440 and includes the image data captured by that imaging device 440. In the illustrated example, the auto-naming user interface 1100 includes twenty preview images 1105. The auto-naming user interface 1100 indicates an identifier status for each imaging device 440. In some embodiments, the identifier status is a graphical indicator or representation of the identifier status. In the illustrated example, the identifier status for each imaging device 440 is indicated by a graphical representation of a box positioned around the corresponding preview image, where a color of the box indicates the identifier status. Following the illustrated example, a first color (i.e., red, represented in FIGS. 11-13 as a dashed box) indicates that the corresponding imaging device 440 was not associated with an identifier (e.g., a non-identified status) while a second color (e.g., green, represented in FIGS. 11-13 as a solid box) indicates that the imaging device 440 was associated with an identifier (e.g., an identified status). As illustrated in FIG. 11, dashed boxes are positioned around the preview images 1105A, 1105F, 1105L, 1105M, 11050, 1105P, 1105S, and 1105T, indicating that the preview images 1105A, 1105F, 1105L, 1105M, 11050, 1105P, 1105S, and 1105T are associated with a non-identified status. As also illustrated in FIG. 11, solid boxes are positioned around the preview images 1105B-1105E, 1105G-1105K, 1105N, and 1105Q-1105R, indicating that the preview images 1105B-1105E, 1105G-1105K, 1105N, and 1105Q-1105R are associated with an identified status.

Although the identifier status is indicated in FIG. 11 by varying a color of a box positioned around a preview image, it should be understood that in some embodiments, the identifier status of an imaging device 440 may be graphically represented in an additional or different way. As one example, the box positioned around the preview image may have a different dashed pattern, a different weight, a different animation, or another type of formatting property. As another example, rather than a box positioned around the preview image, the preview image may be associated with a textual indicator or label indicating the identifier status (e.g., a “Non-Identified” label and/or an “Identified” label). As another example, the preview image may by associated with an animation property indicating the identifier status, such as, a pulsing animation property, a flashing animation property, etc. As yet another example, the preview image may be associated with a graphical symbol or representation indicating the identifier status, such as, a checkmark indicating an identified status and an “x” indicating a non-identified status.

As illustrated in FIG. 11, the auto-naming user interface 1100 also includes a “Setup Readers” button 1106 and a “Trigger” button 1107. The “Setup Readers” button 1106 may be used to adjust one or more settings associated with one or more imaging devices 440, such as, e.g., a light setting. The “Trigger” button 1107 may be used to control the imaging device(s) 440 to capture image data. As one example, where the image data collected was not sufficient (e.g., blurry, or otherwise unsatisfactory), a user may interact with the “Trigger” button 1107 to capture new image data (e.g., as a second image capture of the same target). As another example, the machine vision system of the tunnel may span a distance such that at least a subset of imaging devices do not capture image data related to the calibration target, e.g., because the calibration target has not yet entered a field of view of the subset of imaging devices. In such situations, the calibration target may be advanced (via, e.g., actuating the support structure 450) into the field of view of the subset of imaging devices. Once the calibration target is in the field of view of the subset of imaging devices, a user may interact with the “Trigger” button 1107 to control the subset of imaging devices to capture image data associated with the calibration target now that the calibration target is in the field of view of the subset of imaging devices.

Alternatively, or in addition, in some embodiments, each preview image 1105 is associated with a set of graphical icons, where each graphical icon, when interacted with by a user, performs a function associated with the imaging device 440 (or preview image 1105). As one example, a graphical icon may include a magnifying glass that, when interacted with by a user, zooms in or out on the preview image 1105. As another example, a graphical icon may include a flashlight icon that, when interacted with by a user, may flash an indicator light on the imaging device 440 associated with the preview image 1105 such that a user may easily identify the imaging device 440 in the installed/assembled machine vision system.

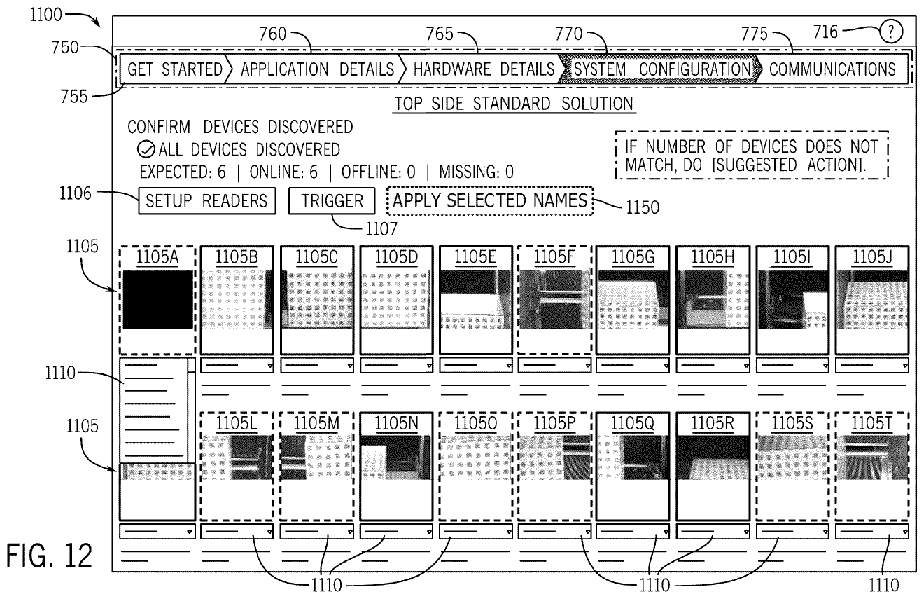

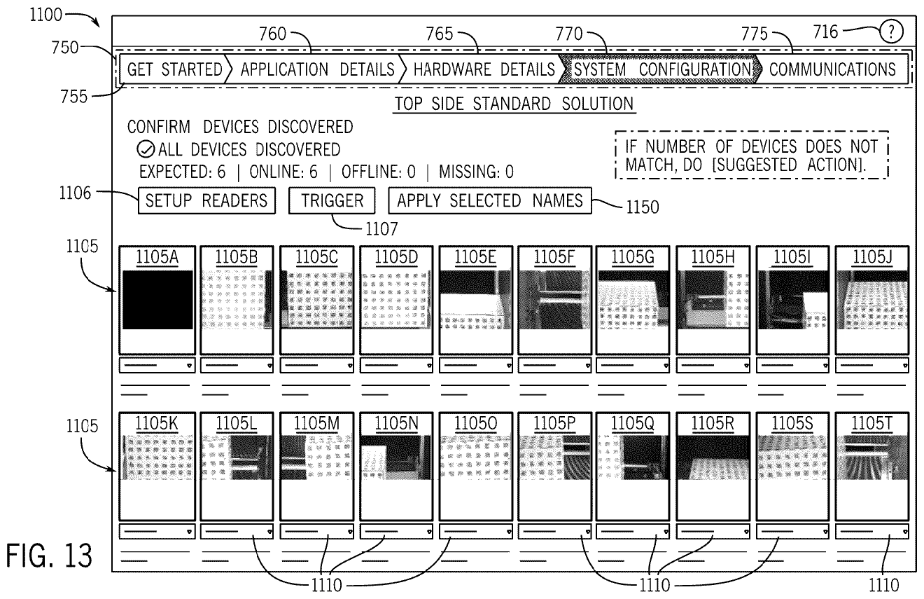

As illustrated in FIG. 11, the auto-naming user interface 1100 includes a set of drop-down menus 1110, where each drop-down menu 1110 is associated with a preview image 1105. The drop-down menus 1110 may include a listing of identifiers associated with the imaging devices 440 of the tunnel (or machine vision system thereof) being commissioned. In some embodiments, when the imaging device 440 is associated with an identified status, the corresponding identifier associated with that imaging device 440 may be automatically selected from the drop-down menu 1110 associated with that imaging device 440. However, a user may alter the automatically selected identifier by interacting with the drop-down menu 1110 and selecting a different identifier for association with the imaging device 440. Alternatively, or in addition, as noted above, in some embodiments, the electronic processor 500 determines a list of identifiers as a list of suggested or remaining identifiers for each imaging device 440 with a non-identified status, as described in greater detail above. As one example, as illustrated in FIG. 11, the imaging device 440 associated with the preview image 1105A has a non-identified status. Accordingly, as illustrated in FIG. 12, a user may interact with a drop-down menu 1110 associated with the preview image 1105A to select an identifier from a list of identifiers included in the drop-down menu 1110. After selecting an identifier from a drop-down menu 1110, the graphical indicator or representation indicating the identifier status of the imaging device 440 may be updated to reflect an identified status (as illustrated in FIG. 12 by the altered color of the box positioned around the preview image 1105A in comparison to FIG. 11). A user may interact with one or more drop-down menus 1110 in order to ensure that each imaging device 440 is associated with a corresponding identifier. As one example, a user may interact with each drop-down menu 1110 associated with an imaging device 440 having a non-identified status until each imaging device 440 is associated with a corresponding identifier, as illustrated in FIG. 13. Accordingly, in some embodiments, the electronic processor 500 may receive one or more user-selected identifiers for one or more of the imaging devices 440 based on user interaction with the auto-naming user interface 1100.

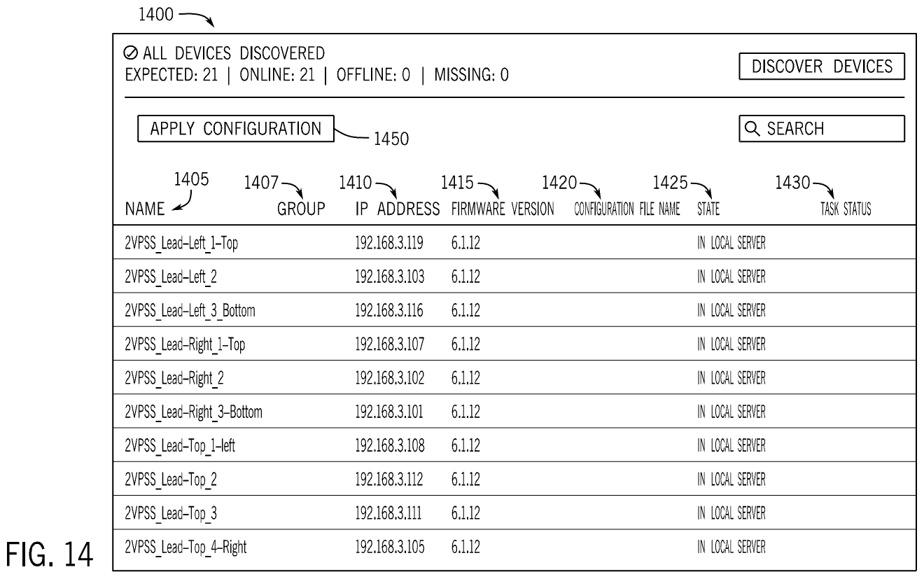

After each imaging device 440 is associated with a corresponding identifier (as illustrated in FIG. 13), a user may interact with an “Apply Selected Names” button 1150 indicating that the user confirms the corresponding identifiers for each imaging device 440. In response to the user interaction with the “Apply Selected Names” button 1150, the electronic processor 500 may generate and provide a device configuration user interface 1400 as illustrated in FIG. 14. As illustrated in FIG. 14, the device configuration user interface 1400 includes an imaging device listing 1405 including each imaging device 440. The device configuration user interface 1400 may also include additional information associated with each imaging device 440, such as, e.g., a group listing 1407, an IP address listing 1410, a firmware version listing 1415, a configuration file name 1420, a status listing 1425, a task status listing 1430, and the like. As also illustrated in FIG. 14, the device configuration user interface 1400 may include an “Apply Configurations” button 1450. A user may interact with the “Apply Configurations” button 1450 to apply device configurations to each imaging device 440.

In some configurations, the device configuration user interface 1400 may be included as a portion of another user interface described herein (e.g., such as the system configuration or auto-naming user interface 1100). For instance, in some configurations, the device configuration user interface 1400 may be associated with a system configuration stage of the tunnel commissioning process. Alternatively, in other configurations, the device configuration user interface 1400 may be associated with a separate or additional stage of the tunnel commissioning process (e.g., as a device configurations stage) and, e.g., the commissioning timeline 750 may include a corresponding “device configurations” indicator.

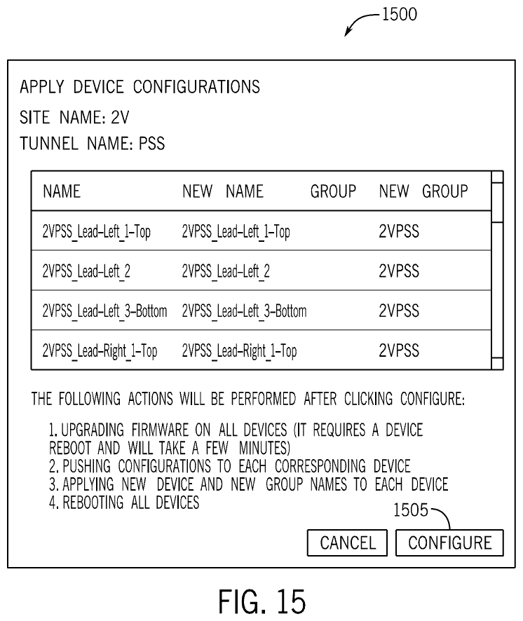

For example, returning to FIG. 6, the electronic processor 500 may configure each imaging device 440 (at block 620). The electronic processor 500 may configure each imaging device 440 based on the corresponding identifier, the tunnel commissioning data, or a combination thereof. As one example, by knowing the corresponding identifier associated with the imaging device 440, the electronic processor 500 may identify a technical setting(s), configuration file(s), firmware file(s), and the like associated with the imaging device(s) 440 (as specified in the tunnel commissioning data). The electronic processor 500 may configure the imaging device(s) 440 (i.e., transition the imaging device(s) 440 from initial settings to operational status) using the technical settings, configuration files, firmware files, and the like, which were identified based on the corresponding identifier for the imaging device 440. Accordingly, in some embodiments, the electronic processor 500 simultaneously configures multiple imaging devices 440. However, as noted herein, in some configurations, the electronic processor 500 may configure a single imaging device 440 using the methods and systems described herein. In some embodiments, prior to configuring the imaging device(s) 440 (at block 620), the electronic processor 500 prompts the user for confirmation via a confirmation dialogue box 1500, as illustrated in FIG. 15. In response to the user interacting with a “Configure” button 1505 of the confirmation dialogue box 1500, the electronic processor 500 may then initiate the configuration process of the imaging device(s) 440 (at block 620).

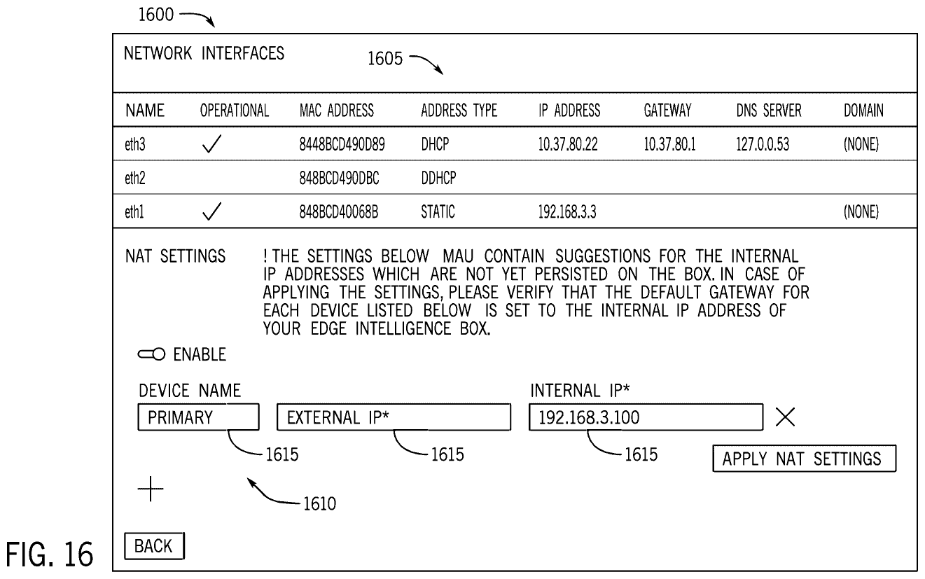

In some embodiments, the electronic processor 500 generates and provides a network configuration user interface 1600 as illustrated in FIG. 16. The network configurations user interface 1600 provides information relevant to the network configuration(s) associated with each of the one or more imaging devices 440. As illustrated in FIG. 16, the network configuration user interface 1600 includes a network interfaces table 1605. The network interfaces table 1605 provides information related to, e.g., a network interface name, an operational status, a MAC address, an address type, an IP address, a subnet mask, a gateway, a DNS server, a domain, and the like. The network configuration user interface 1600 also includes a network address translation (“NAT”) settings portion 1610. The NAT settings portion 1610 includes a set of input mechanisms 1615 that enables a user to provide additional NAT settings. As illustrated in FIG. 16, the set of input mechanisms 1615 includes a set of text fields for receiving a device name, an external IP address, and an internal IP address. In some embodiments, the network configurations user interface 1600 enables a user to customize final network settings, including, e.g., NAT entries for the system. In some configurations, the network configurations user interface 1600 may be included as a portion of another user interface described herein (e.g., such as the system configuration or auto-naming user interface 1100). For instance, in some configurations, the network configurations user interface 1600 may be associated with a system configuration stage of the tunnel commissioning process. Alternatively, in other configurations, the network configurations user interface 1600 may be a separate stage of the tunnel commissioning process (e.g., as a network configurations stage) and, e.g., the commissioning timeline 750 may include a corresponding “network configurations” indicator.

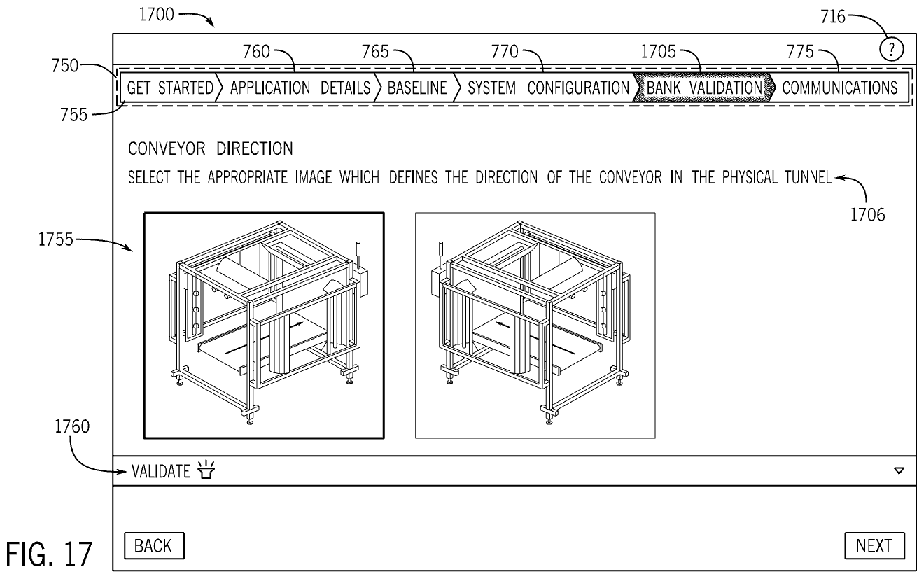

In some embodiments, the electronic processor 500 may generate a bank validation user interface 1700, as illustrated in FIGS. 17-21. In some embodiments, the commissioning process may include a bank validation stage. Accordingly, as illustrated in FIG. 17, the bank validation user interface 1700 includes a “bank validation” identifier 1705 in the commissioning timeline 750. As also illustrated in FIG. 17, the bank validation user interface 1700 can include a conveyor direction portion 1755 and a validate portion 1760. The conveyor direction portion 1755 can prompt a user to indicate a direction of travel associated with a conveyor (e.g., the conveyor 116) of the tunnel being commissioned. As one example, as illustrated in FIG. 17, the conveyor direction portion 1755 includes graphical representations of directions of travel associated with a conveyor. A user may interact with the graphical representations (e.g., click with a mouse) to select a direction of travel associated with a conveyor of the tunnel being commissioned. Alternatively, or in addition, in some embodiments, the conveyor direction portion 1755 can include additional, fewer, or different mechanisms for a user to provide a direction of travel associated with a conveyor of the tunnel being commissioned (e.g., radio buttons associated with various directions of travel).

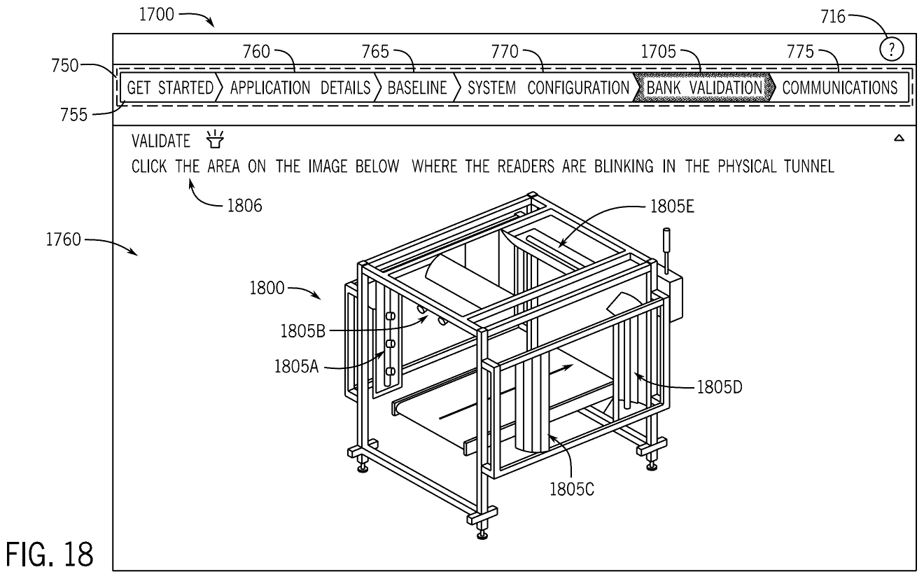

After a user selects a direction of travel associated with a conveyor of the tunnel being commissioned, the commissioning process may progress to a bank validation stage of the bank validation step of the commissioning process. As part of the bank validation stage of the bank validation step of the commissioning process, the validate portion 1760 of the bank validation user interface 1700 can be expanded, as illustrated in FIGS. 18-21. As illustrated in FIG. 18, the validate portion 1760 may provide a graphical representation 1800 of the tunnel being commissioned. The graphical representation 1800 may include a set of banks. In the example illustrated in FIGS. 18-21, the graphical representation 1800 includes a first bank 1805A, a second bank 1805B, a third bank 1805C, a fourth bank 1805D, and a fifth bank 1805E. In some embodiments, the electronic processor 500 generates the graphical representation 1800 based on specifications associated with the tunnel being commissioned (e.g., based on the tunnel commissioning data 570). Although the graphical representation 1800 of FIG. 18 includes multiple banks, it should be understood that in some configurations, the graphical representation 1800 may include a single bank, including, e.g., a single bank with a single imaging device.

As illustrated in FIG. 18, the validate portion 1760 includes instructions to a user indicating how to validate the set of banks (represented in FIG. 18 by reference numeral 1806). In the illustrated example of FIG. 17, the instructions 1706 instruct a user to “Click the area on the image below where the readers are blinking in the physical tunnel.” Accordingly, while observing the physical tunnel, the user clicks a bank that is blinking.

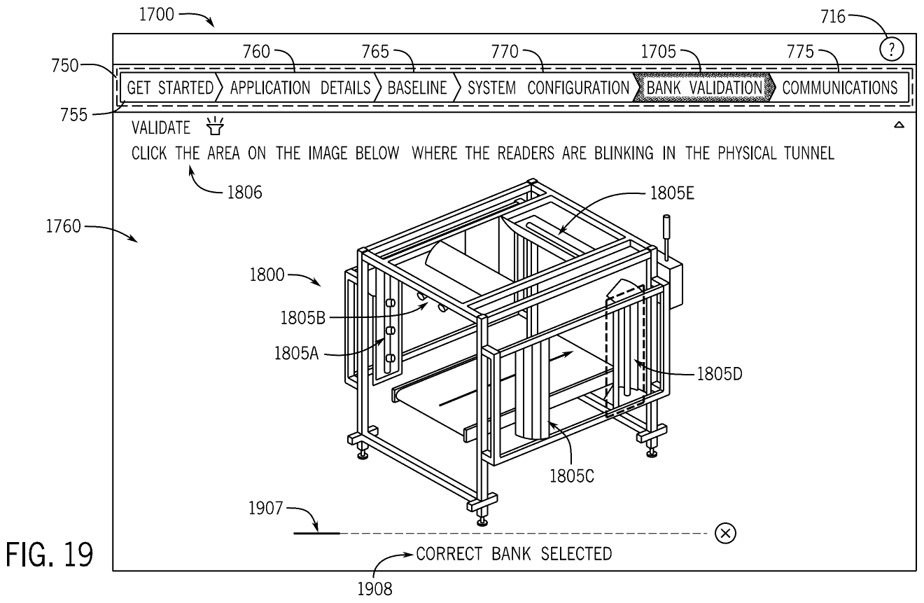

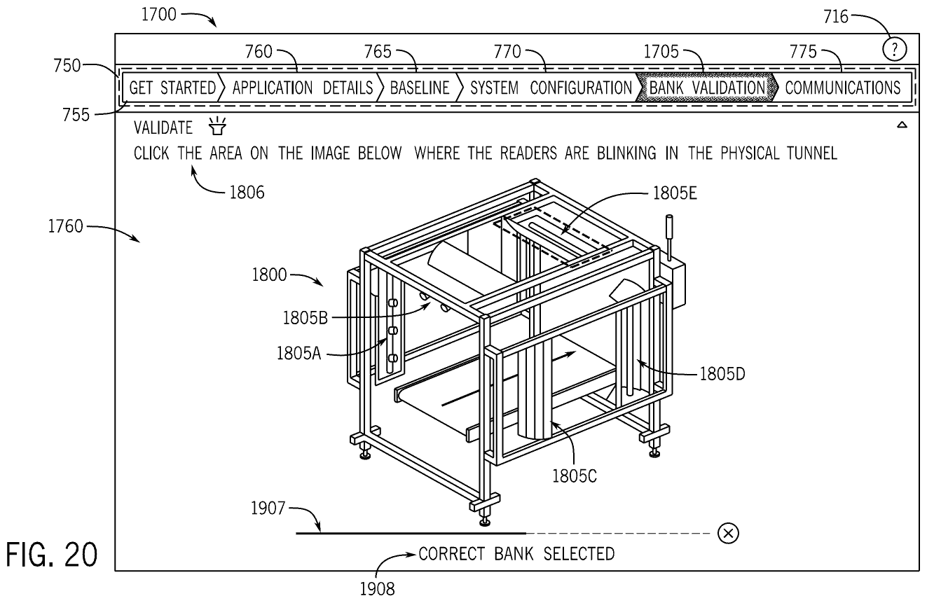

FIGS. 19 and 20 illustrate a user interacting with the graphical representation 1800 based on which bank is blinking on the physical tunnel. As one example, as illustrated in FIG. 19, the user selects the fourth bank 1805D in response to observing that the imaging devices associated with the fourth bank 1805D are blinking on the physical tunnel. As another example, as illustrated in FIG. 20, the user selects the fifth bank 1805E in response to observing that the imaging devices associated with the fifth bank 1805E are blinking on the physical tunnel.

As also illustrated in FIGS. 19-20, the validate portion 1760 can also include a progress indicator 1907 (illustrated in FIGS. 19-20 as a progress bar). The progress indicator 1907 may provide a graphical representation or indication of a progress status associated with validating the banks of a tunnel being commissioned. Additionally, in some embodiments, the validate portion 1760 can also provide a confirmation indication (represented in FIGS. 19-20 by reference numeral 1908) with respect to a user interacting with the graphical representation. For example, when the user correctly selects a bank on the graphical representation 1800 that is associated with the bank of the physical tunnel that is blinking, the validate portion 1760 may indicate that a user selected the correct bank.

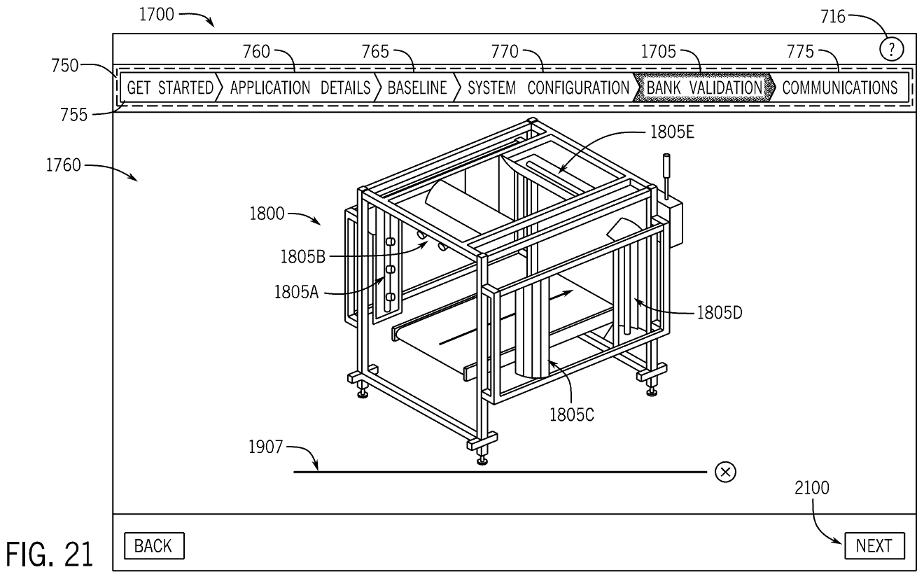

After validating each bank included in the set of banks, the bank validation user interface 1700 can prompt the user to navigate to the next stage of the commissioning process. In some embodiments, as illustrated in FIG. 21, the bank validation user interface 1700 can prompt the user by activating a “Next” button 2100, such that a user can navigate to the next stage of the commissioning process.

As noted above, the commissioning timeline 750 is one non-limiting example of a commissioning timeline and, in some embodiments, the commissioning process (e.g., the commissioning timeline 750) includes additional, different, or fewer stages and/or indicators than illustrated. As one example, as illustrated in FIGS. 18-22, in some embodiments, the bank validation stage of the commissioning process can occur between the system configuration stage and the communication stage. However, it should be understood that the order of the stages of the commissioning process described herein is one non-limiting example order of the stages and that the order of the stages of the commissioning process may be different.

Returning to FIG. 6, in some embodiments, the electronic processor 500 also generates and transmits a commissioning report (at block 625). In some embodiments, the commissioning report includes information associated with the commissioning process. For example, the commissioning report includes the tunnel commissioning data, the set of commissioning parameters, the set of methodology details, information included in the device configuration user interface 1400 (e.g., the imaging device listing 1405 including each imaging device 440, the group listing 1407, the IP address listing 1410, the firmware version listing 1415, the configuration file name 1420, the status listing 1425, the task status listing 1430, etc.), information included in the network configuration user interface 1600 (e.g., the network interfaces table 1605), and the like. In some embodiments, the electronic processor 500 generates and transmits the commissioning report to a remote device, such as, e.g., the user device 415 for display to a user via the HMI 580, a human machine interface associated with the tunnel subsystem 405, a remote database, and the like. Alternatively, or in addition, in some embodiments, the electronic processor 500 stores the commissioning report in the memory 505. In some configurations, the commissioning report may be displayed to the user as part of the communication stage of the commissioning process.

As noted herein, the electronic processor 500 may execute the application 560 to commission a tunnel, and, more specifically, commission a tunnel by identifying one or more imaging devices (e.g., the imaging device(s) 440) associated with a tunnel. In some instances, the electronic processor 500 may execute the application 560 to automatically identify one or more imaging devices (e.g., the imaging device(s) 440), such as part of an auto-naming process (e.g., as described herein with respect to FIG. 6). Alternatively, or in addition, in some configurations, the electronic processor 500 may execute the application 560 to facilitate manual identification of one or more imaging devices (e.g., the imaging device(s) 440) based on user input, as part of a manual naming process.

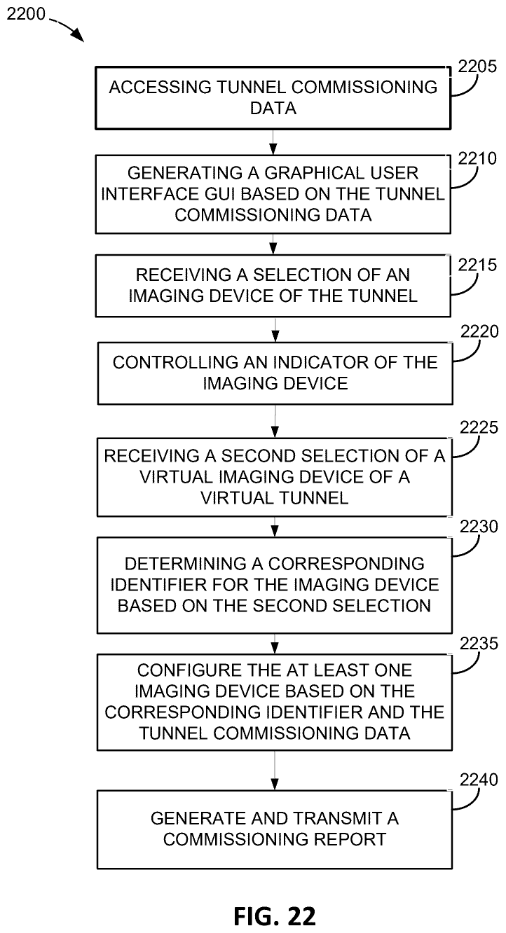

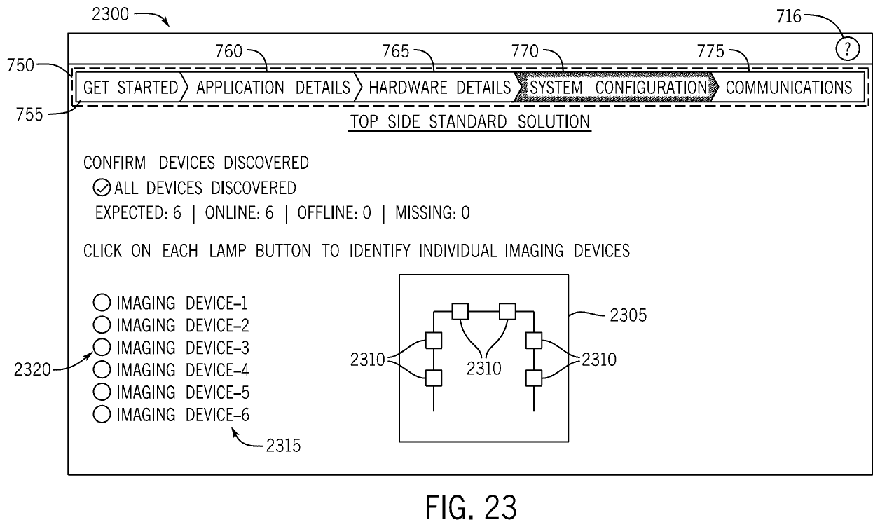

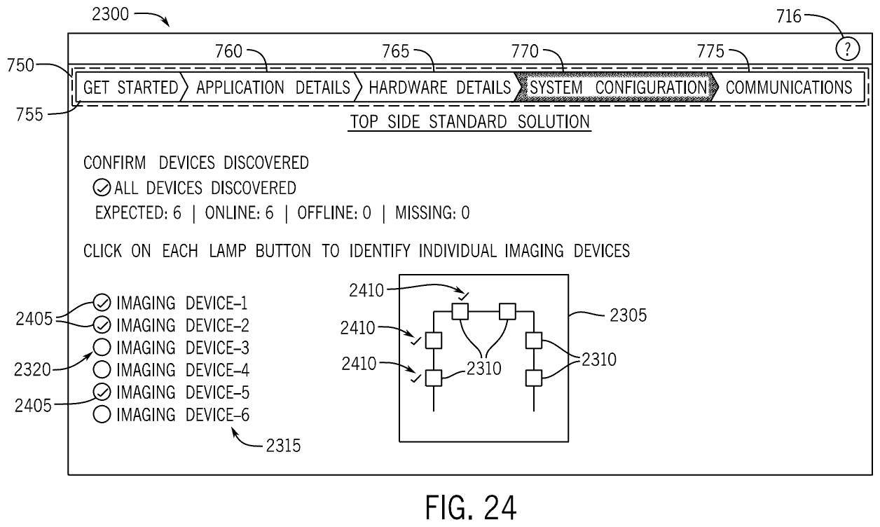

FIG. 22 is a flowchart illustrating a method 2200 for commissioning a machine vision system for a tunnel using a manual naming process for identifying one or more imaging devices associated with the tunnel according to some embodiments. The method 2200 is described herein as being performed by the server 410 and, in particular, the application 560 as executed by the electronic processor 500. However, as noted above, the functionality described with respect to the method 2200 may be performed by other devices, such as the user device 415, component(s) of the tunnel subsystem 405, or distributed among a plurality of devices, such as a plurality of servers included in a cloud service. The method 2200 is described herein with respect to FIGS. 23-24. FIGS. 23-24 provide an example user interface associated with a manual naming process according to some configurations.

As illustrated in FIG. 22, the method 2200 includes accessing, with the electronic processor 500, the tunnel commissioning data 570 (at block 2205). In some configurations, the electronic processor 500 accesses the tunnel commissioning data 570 as similarly described herein with respect to block 605 of the method 600 illustrated in FIG. 6.

In some configurations, the electronic processor 500 may generate a graphical user interface (GUI) (at block 2210). The GUI may include a graphical representation of a virtual tunnel representing the tunnel being commissioned. The electronic processor 500 may transmit the GUI for display of the GUI to a user. For instance, in some configurations, the electronic processor 500 may generate and transmit the GUI to a remote device, such as, e.g., the user device 415 for display to a user via the HMI 580, a human machine interface associated with the tunnel subsystem 405, a remote database, and the like.

FIG. 23 illustrates an example GUI 2300 associated with a manual naming process according to some configurations. As illustrated in FIG. 23, the GUI 2300 may include a graphical representation of a virtual tunnel 2305. The virtual tunnel 2305 may represent the physical tunnel (or machine vision system thereof) being commissioned. The virtual tunnel 2305 may include one or more virtual imaging devices 2310. The virtual imaging devices 2310 may represent the imaging devices 440 included in the physical tunnel being commissioned. In some configurations, a virtual imaging device 2310 is positioned on the virtual tunnel 2305 at a location that corresponds to a location of the corresponding imaging device 440 on the physical tunnel being commissioned. For example, when the physical tunnel being commissioned includes three imaging devices, then the virtual tunnel 2305 may include three virtual imaging devices 2310, where each of the virtual imaging devices 2310 are positioned on the virtual tunnel 2305 at locations corresponding to the actual locations of the three imaging devices on the physical tunnel being commissioned. In some configurations, the electronic processor 500 accesses the tunnel specification package and generates the virtual tunnel 2305, the virtual imaging device(s) 2310, or a combination thereof based on the data included in the tunnel specification package.

As illustrated in FIG. 23, the GUI 2300 may also include an interactive list 2315 of the one or more imaging devices 2310. Each imaging device 2310 included in the interactive list 2315 includes a checkbox 2320 (as a GUI element). A user may interact with the interactive list 2315 by selecting an imaging device included in the interactive list 2315. In some configurations, the user may select an imaging device by interacting with (e.g., selecting) the checkbox 2320 associated with the imaging device. Alternatively, or in addition, in some configurations, the user may select the imaging device by interacting with (e.g., clicking with a mouse) the text for the imaging device. In such configurations, the text included in the interactive list 2315 may be selectable.

Returning to FIG. 22, the electronic processor 500 may receive a selection of an imaging device of the tunnel (at block 2215). In some configurations, the electronic processor 500 receives the selection of an imaging device as a user interaction with the GUI 2300. For instance, a user may select an imaging device by interacting with an imaging device included in the interactive list 2315. A user may select an imaging device from the interactive list 2315 by selecting a checkbox 2320 associated with the imaging device. Alternatively, or in addition, the user may select an imaging device by selecting the text included in the interactive list 2315.

In response to receiving the selection of an imaging device of the tunnel via the GUI 2300 (at block 2215), the electronic processor 500 may control an indicator of the imaging device (at block 2220). In some configurations, the electronic processor 500 may determine a physical imaging device 440 included in the tunnel being commissioned that is associated with the selection from the GUI 2300. The electronic processor 500 may then generate and transmit a control signal to the corresponding imaging device 440 of the tunnel being commissioned. In response to receiving the control signal, the corresponding imaging device 440 may provide an indication to a user. In some examples, the imaging device 440 includes a visual indicator, such as a light or LED. When the imaging device 440 receives the control signal, the visual indicator is controlled to provide an indication (e.g., a visual indication). In some instances, the visual indicator may blink or flash in response to the control signal. Alternatively, or in addition, the visual indicator may light up and remain lit in response to the control signal.

The electronic processor 500 may receive a second selection via the GUI 2300 of a virtual imaging device (at block 2225). The second selection may be a user interaction with the graphical representation of the virtual tunnel 2305, including, e.g., a virtual imaging device 2310 thereof. For example, the second selection may be a user interaction with one of the virtual imaging devices 2310 of the virtual tunnel 2305. Accordingly, in some configurations, the graphical representation of the virtual tunnel 2305 (including the virtual imaging devices 2310) may be interactive or selectable such that a user may select one or more of the virtual imaging devices 2310.

For example, a user may visually observe which of the physical imaging devices 440 is providing the visual indication (e.g., which visual indicator is lit and/or flashing). The user may then select the corresponding virtual imaging device 2310 of the virtual tunnel 2305 that corresponds to the physical imaging device 440 providing the visual indication. Accordingly, in some configurations, the user may identify which physical imaging device 440 is providing the visual indication and match that physical imaging device 440 to a corresponding virtual imaging device 2310 in the graphical representation of the virtual tunnel 2305, where a location of the virtual imaging device 2310 in the graphical representation of the virtual tunnel 2305 selected by the user corresponds to a location of the physical imaging device 440 on the tunnel that is providing the visual indication.

The electronic processor 500 may determine a corresponding identifier for the imaging device 440 based on the second selection (at block 2230). In some configurations, the electronic processor 500 determines the corresponding identifier for the imaging device 440 by associating the imaging device 440 selected from the interactive list 2315 with the virtual imaging device 2310 identified in the second selection (e.g., the virtual imaging device 2310 selected in the graphical representation of the virtual tunnel 2305). In some configurations, the electronic processor 500 determines the corresponding identifier for the imaging device 440 (at block 2230) as similarly described herein with respect to block 615 of the method 600 illustrated in FIG. 6.

In some configurations, after determining the corresponding identifier for the imaging device 440 (at block 2230), the electronic processor 500 may update the GUI 2300 (or otherwise indicate) that the imaging device 440 is associated with a corresponding identifier. In some configurations, the electronic processor 500 may update the GUI 2300 to include a mark or graphical indicator to indicate the association. Alternatively, or in addition the electronic processor 500 may update the GUI 2300 by altering or modifying an existing element or component. For example, in some configurations, the electronic processor 500 may alter (or add) a formatting property (e.g., a color, a font, a font style, a transparency, etc.) to indicate the association. As another example, in some configurations, the electronic processor 500 may alter (or add) a display feature or property (e.g., an animation feature, etc.) to indicate the association. For example, as illustrated in FIG. 24, the electronic processor 500 may generate and provide a checkmark 2405 proximate to the imaging device(s) 440 selected from the interactive list 2315, where the checkmark 2405 indicates that the imaging device(s) 440 are associated with a corresponding identifier. Alternatively, or in addition, as also illustrated in FIG. 24, the electronic processor 500 may generate and provide a checkmark 2410 proximate to the virtual imaging device(s) 2310 in the graphical representation of the virtual tunnel 2305, where the checkmark 2410 indicates that the virtual imaging device(s) 2310 are associated with a corresponding identifier.

In some configurations, the electronic processor 500 may receive a selection (e.g., as a user interaction with the GUI), where the selection is a request to disassociate an imaging device 440 with a corresponding identifier. For instance, in some configurations, a user may interact with the GUI 2300 by unselecting an imaging device 440 included in the interactive list 2315 by, e.g., interacting with a checkbox 2320. By interacting with the corresponding checkbox 2320, after the imaging device 440 associated with that checkbox 2320 has been associated with a corresponding identifier, the electronic processor 500 may disassociate the corresponding identifier from the imaging device 440 included in the interactive list 2315.

In some configurations, the electronic processor 500 may repeat one or more steps included in the method 2200. For instance, in configurations where the tunnel being commissioned includes multiple imaging devices, the electronic processor 500 may repeat one or more steps of the method 2200 for one or more of the multiple imaging devices. In some instances, the electronic processor 500 may repeat blocks 2215-2230 for each of the multiple imaging devices and then perform blocks 2235 and 2240 of FIG. 22, as described in greater detail herein, once each imaging device is associated with a corresponding identifier.

As illustrated in FIG. 2200, the electronic processor 500 may configure the imaging device(s) 440 based on the corresponding identifier and the commissioning data (at block 2235) and may generate and transmit a commissioning report (at block 2240). In some configurations, the electronic processor 500 may configure the imaging device(s) 440 (at block 2235) as similarly described herein with respect to block 620 of the method 600 illustrated in FIG. 6. In some configurations, the electronic processor 500 may generate and transmit a commissioning report (at block 2240) as similarly described herein with respect to block 625 of the method 600 illustrated in FIG. 6.

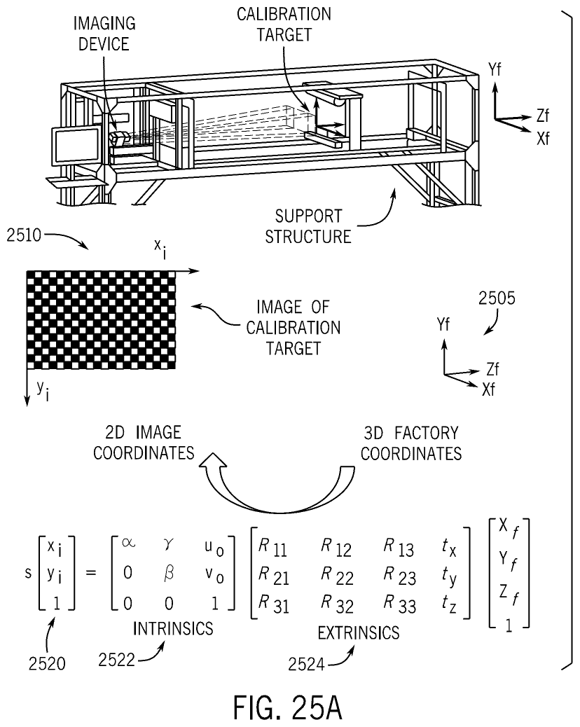

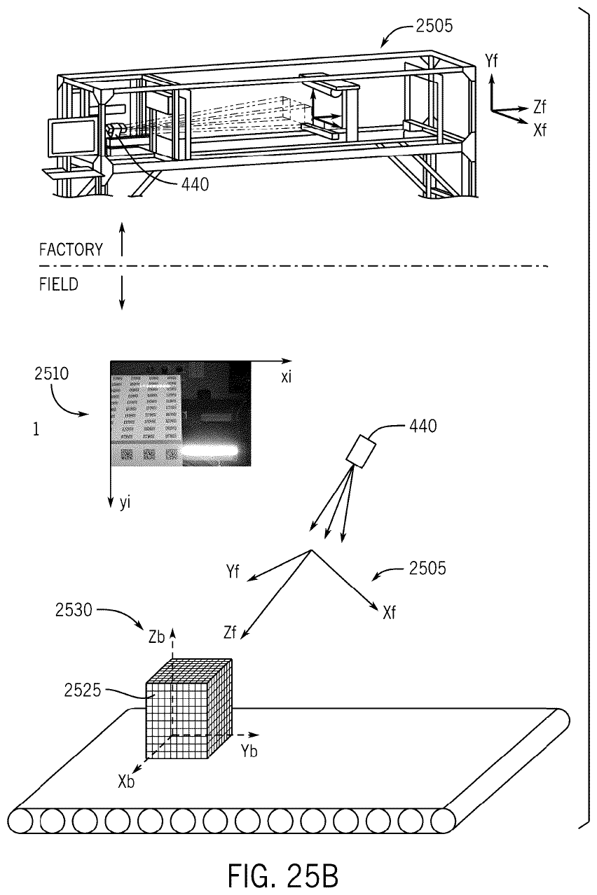

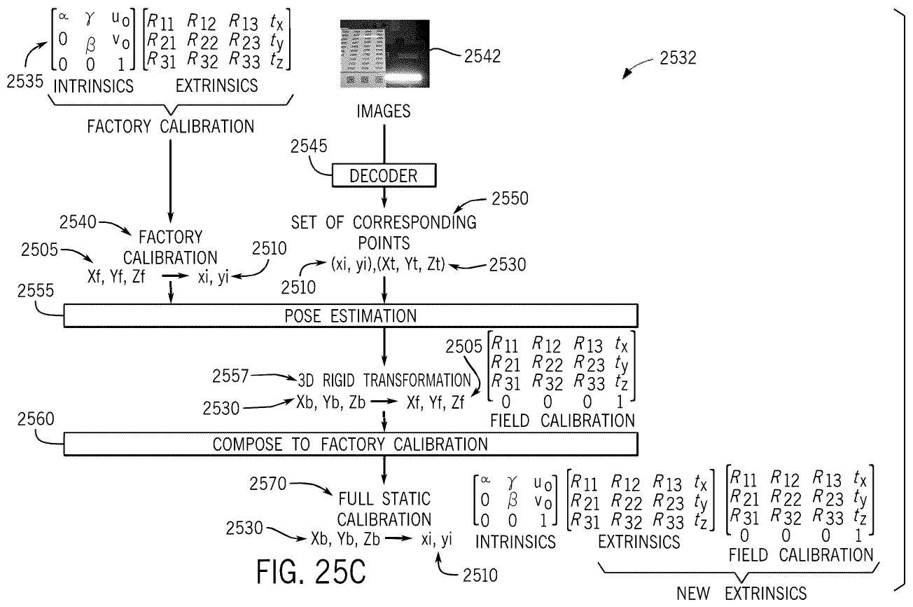

FIG. 25A illustrates an example of a factory calibration setup that can be used to find a transformation between a 2D image coordinate space and a 3D factory (or camera) coordinate space (e.g., as implemented during manufacturing of the camera using a standard calibration target). As illustrated in FIG. 25A, an imaging device can generate images that project points in a 3D factory coordinate space (Xf, Yf, Zf) (represented in FIG. 25A by reference numeral 2505) onto a 2D image coordinate space (xi, yi) (represented in FIG. 25A by reference numeral 2510). The 3D factory coordinate space 2505 can be defined, e.g., based on a support structure (which may sometimes be referred to as a fixture) that supports a calibration target at various known locations relative to a mount for the imaging device. Images of the calibration target (e.g., at different known locations) can be used to find the transform between the factory coordinate space 2505 and the image coordinate space 2510.

The transformation between the factory coordinate space 2505 and the image coordinate space 2510 may be represented by a transformation 2520 of FIG. 25A. The transformation 2520 illustrated in FIG. 25A is a representative transformation. Accordingly, the transformation between the factory coordinate space 2505 and the image coordinate space 2510 may be represented by a different transformation than the transformation 2520 of FIG. 5A (including, e.g., a more complex transformation). As illustrated in FIG. 25A, the transformation 2520 includes intrinsic parameters (or “intrinsics”) 2522 and extrinsic parameters (or “extrinsics”) 2524. The intrinsic parameters 2522 can represent parameters that relate pixels of the image sensor of the imaging device 440 to an image plane of the imaging device 440 based on intrinsic properties of the camera, such as, e.g., a focal length, an image sensor format, a principal point, lens distortion, etc. The extrinsic parameters 2524 can represent parameters that relate points in 3D common coordinates (e.g., with an origin defined by a target used during factory calibration) to 3D camera coordinates (e.g., with a camera center defined as an origin). Thus, as represented in FIG. 25A, a factory calibration can provide a transformation between 3D factory coordinates and 2D image coordinates based on intrinsic parameters of the camera and extrinsic parameters associated with the camera and the relative 3D space.

Generally, the overall camera calibration process goal is to find a transformation between a physical 3D coordinate space (e.g., in mm) and the image 2D coordinate space (e.g., in pixels). The transformation 2520 of FIG. 25A illustrates an example for such a transformation using a simple pinhole camera model. The transformation 2520 can have other nonlinear components (e.g., to represent lens distortion). The transformation 2520 can be split into extrinsic and intrinsic parameters. The extrinsics can depend on the location and orientation of mounting the imaging device(s) with respect to the physical 3D coordinate space. The intrinsics can depend on internal imaging device parameters, such as, e.g., the sensor and lens parameters. The calibration process goal is to find value(s) for these intrinsic and extrinsic parameters. In some embodiments, the calibration process can be split into two parts: one part executed in the factory calibration and another part executed in the field.

Additionally, the main goal of factory calibration is to calculate intrinsics, which do not change based on mounting the imaging device. This can simplify the process in the field to finding extrinsics after mounting the imaging device, which makes the process in the field during the system installation much faster and easier.

Once a camera has been factory calibrated (i.e., the relevant intrinsics and extrinsics for the camera determined relative to factory space as described above), it may still be necessary to calibrate the camera relative to the installed environment to ensure that images acquired using the camera can be appropriately analyzed. In some implementations, a factory calibration can thus be followed by a field calibration that can determine a transform between the factory calibrate coordinate system of the camera and a reference coordinate system of the installation site (e.g., as part of initial set-up for a tunnel for a transport system that includes the camera).

In this regard, FIG. 25B illustrates example coordinate spaces associated with various portions of a system for capturing one or more images of one or more sides of a calibration target 2525 and associating an identifier to at least one imaging device (e.g., the imaging device(s) 440) in accordance with an embodiment of the technology. In particular, FIG. 25B illustrates the factory coordinate space 2505, the image coordinate space 2510, and an object coordinate space (Xb, Yb, Zb) for the calibration object 2525 (represented in FIG. 25B as 2530). Generally, the object coordinate space 2530 can be defined based on an object (e.g., the calibration target 2525) being used to perform the calibration (e.g., a field calibration process). As illustrated in FIG. 25B, the imaging device 440 (and thus the factory coordinate space 2505) can be repositioned such that the factory coordinate space 2505 can be particularly oriented relative to the object coordinate space 2530 (e.g., as supported within a predefined range of locations on a tunnel support structure).

Additionally, in some embodiments, during a calibration process (e.g., a field calibration process), an object coordinate space (Xb, Yb, Zb) can be defined based on an object being used to perform the calibration. As one example, as illustrated in FIG. 25B, symbol(s) can be placed onto an object, where each symbol is associated with a particular location in object coordinate space. Images of the calibration target 2525 can then be acquired using the camera, and a transformation can be determined between the factory coordinate space 2505 and the object coordinate space 2530. Of note, although the illustrated calibration does not necessarily fix the object coordinate space 2530 relative to another frame (e.g., a coordinate space defined by a fixed origin location on a conveyor), some calibration procedures can include calculations for further transformations from the object coordinate space 2530 to another (e.g., transport) coordinate space (not shown) as part of a calibration process.

In some cases, a particular calibration object can facilitate relatively easy identification of particular locations in the object coordinate space 2530 within the image coordinate space 2510, as in turn can be specified relative to the factory calibration space 2505 by factory calibration (e.g., as discussed above). As one example, as illustrated in FIG. 25B, symbol(s) can be placed onto the calibration target 2525, wherein each symbol is associated with a particular location in the object coordinate space 2530. Thus, for example, upon decoding or analyzing a particular symbol within an image (e.g., any or all of the symbols shown relative to the image coordinate space 2510 in FIG. 25B), a correspondence can be determined between a particular pixel or pixel area in the image coordinate space 2510 and a particular location in the object coordinate space 2530 (e.g., a location corresponding to a top right corner on a leading face of the object 2525, a location corresponding to a leading edge center point of a top face of the object 2525, etc.). Based on these identified locations within the image coordinate space 2510 and using known (or derivable) dimensions of the object 2525, a transform between the object coordinate space 2530 and the image coordinate space 2510 can thus be determined (via the factory coordinate space 2505) and the calibration of the image coordinate space 2510 to the object coordinate space 2530 thereby achieved.

Further in this regard, FIG. 25C illustrates a more detailed example of a process 2532 for generating an imaging device model. In some embodiments, the imaging device model may be used as part of the method 600 of FIG. 6 (e.g., as part of block 615). As one example, the electronic processor 500 may determine a corresponding identifier for at least one of the imaging devices 440 using the imaging device model. Although not illustrated, in some embodiments, the imaging device model may be stored in the memory 505 of the server 410, a memory of the user device 415, a memory component of the tunnel subsystem 405, and/or another memory device.

The imaging device model may be useable to transform coordinates of the calibration target 2525 in a 3D coordinate space (e.g., the object coordinate space 2530 of FIG. 25B) into coordinates in a 2D coordinate space (e.g., the image coordinate space 2510 of FIGS. 25A-25B) associated with the imaging device 440 in accordance with an embodiment of the technology, including by capturing one or more images of the calibration target 2525 (e.g., one or more images of multiple sides of the calibration target 2525). As described above with respect to FIG. 25A, in some embodiments, an imaging device (e.g., the imaging device 440) can be calibrated before being installed in the field (e.g., a factory calibration can be performed). Such a factory calibration can be used to generate an initial camera model that can be used to map points in 3D factory coordinate space (e.g., the factory coordinate space 2505) to 2D points in the image coordinate space 2510.

As one example, as illustrated in FIG. 25C and also discussed above, a factory calibration process can be performed to generate extrinsic parameters that can be used with intrinsic parameters to map points in the 3D factory coordinate space (e.g., the factory coordinate space 2505) into 2D points in the image coordinate space. (e.g., the image coordinate space 2510). The factory calibration process is represented in FIG. 25C by reference numeral 2535 and the transformation (or mapping of points) between the factory coordinate space 2505 into the image coordinate space 2510 is represented in FIG. 25C by reference numeral 2540.

As noted above, with reference to FIG. 25B, symbol(s) can be placed onto the calibration target 2525, where each symbol is associated with a particular location in the object coordinate space 2530. For example, each symbol can encode information indicating a particular location on a calibration target (e.g., a relative location or an absolute location within the coordinate space 2530) and can be placed on the calibration target 2525 with sufficient accuracy that the actual location of the symbol (e.g., of a timing pattern or other feature of the symbol) closely corresponds to the encoded location. Accordingly, as illustrated in FIG. 25C, an image 2542 of the calibration target 2525 can include at least one symbol, and preferably many symbols, wherein the at least one symbol is associated with at least one particular location in the object coordinate space 2530. A decoder 2545 (e.g., of various known types) may be thus implemented to decode the at least one symbol to determine a set of at least one corresponding points in the object coordinate space 2530 (e.g., at least one absolute location within the object coordinate space 2530). Thus, as also discussed above, a transformation between the object coordinate system 2530 and the image coordinate space 2510 can be determined, as represented in FIG. 25C by reference numeral 2550.

As illustrated in FIG. 25C, with a factory calibration and an initial field calibration in place (e.g., as represented by reference numerals 2540, 2550), a pose estimation 2555 can be performed to determine a 3D rigid transformation 2557 of the object coordinate space 2530 to the factory coordinate space 2505, using the common image coordinate space 2510 as a common linking reference frame. In other words, with the mathematical transformations 2540, 2550 having been determined (e.g., as discussed above), one or more image(s) with the common image coordinate space 2510 can be leveraged to determine the transformation 2557 between object coordinate space 2530 and the factory coordinate space 2505. Correspondingly, the specified 3D rigid transformation 2557 can allow the factory calibration to be composed (represented in FIG. 25C by reference numeral 2560), as can result in a full static calibration 2570 under which the object coordinate space 2530 can be transformed to the image coordinate space 2510 (and vice versa).

Accordingly, the symbol(s) of the calibration target 2525 (e.g., a box with codes affixed that define a position of each code in the object coordinate space) can indicate fixed coordinate locations for a calibration object in the object coordinate space 2530, and the location in the object coordinate space 2530 can be correlated with locations in the image coordinate space 2510 of the calibration target 2525 (e.g., relating coordinates in (Xt, Yt, Zt) to (xi, yi)). Such correspondence can be used to update the camera model to account for the transformation between the factory coordinate space 2505 and the object coordinate space 2530, including through the use of a field calibration extrinsic parameter matrix (e.g., as can be defined using the 3D rigid transformation discussed above. The field calibration extrinsic parameter matrix can thus be used in conjunction with the camera model derived during factory calibration to relate points in the object coordinate space 2530 (Xb, Yb, Zb) to points in the image coordinate space 2510 (xi, yi).

In some embodiments, such a transformation can be used to map 3D points of the calibration target 2525 to an image of the calibration target 2525 (e.g., the image 2542), such that a different respective identifier may be associated with each of a plurality of imaging devices arranged to collectively acquire images of a common area (e.g., a tunnel of a transport system). For example, acquisition of multiple images of the calibration object 2525 by multiple imaging devices (e.g., from different perspectives, with the object 2525 in the same location) and a corresponding factory calibration of each of the imaging devices (e.g., as described above) can calibrate each of the imaging devices to a common coordinate frame (i.e., the coordinate space 2530). Thus, the orientation of the imaging devices relative to each other can be determined (via the calibration to the common coordinate frame), even if the object coordinate space 2530 is not itself calibrated to a particular transport system or other on-site reference frame. Correspondingly, a location of the cameras relative to each other can be determined and this relative location can be used for further field set-up operations, including to name or otherwise set up the imaging devices based on their locations relative to each other and a set of predetermined relative locations within a larger system (e.g., a relative location on a tunnel or other organized assembly of cameras).

In calibration processes as generally discussed above, acquisition of multiple images may sometimes be required, so as to ensure that sufficient numbers of imaging devices can be calibrated to a common reference frame (e.g., the coordinate space 2530) via common inclusion of one or more of the same real-world locations (e.g., on the calibration target 2525) in corresponding images by different imaging devices. Similarly, it may be beneficial to position a calibration target to optimize (e.g., maximize) both the number of faces and the number of location codes (or other identifiable features) that will be included in images of multiple imaging devices.

The model depicted in FIGS. 25A, 25B, and 25C is a simplified (e.g., pinhole camera) model that can be used to correct for distortion caused by projection to avoid overcomplicating the description. More sophisticated models (e.g., including lens distortion) can be used in some implementations, in connection with mechanisms described herein, with similar overall operations for field calibration.

Further, the particular operations presented above constitute merely an example calibration process, and other techniques can be used to define a transformation between object coordinate space and image coordinate space. As one example, in some embodiments, each of the imaging devices is associated with (or has) a factory calibration. Alternatively, or in addition, in some embodiments, one or more of the imaging devices are not factory calibrated (e.g., are not associated with a factory calibration). In such embodiments, one or more of the imaging devices can have approximated intrinsics based on, e.g., lens and sensor specifications. As another example, rather than performing a factory calibration and a field calibration, a field calibration can be used to derive a model relating object coordinates to image coordinates. However, this may cause replacement of an imaging device 440 to be more cumbersome, as the entire calibration may need to be performed to use a new imaging device 440. In some embodiments, calibrating an imaging device 440 using a calibration target to find a transformation between a 3D factory coordinate space and image coordinates, and calibrating the imaging device in the field to find a transformation that facilitates mapping between common coordinates (e.g., associated with a conveyor, a support platform, or a dimensioner) can facilitate replacement of an imaging device 440 without repeating the field calibration (e.g., as described in U.S. Pat. No. 9,305,231, issued Apr. 5, 2016, which is hereby incorporated by reference herein in its entirety).

As noted above, calibration under the disclosed method can be improved by increasing the number of locations and faces (or other features) of a calibration target that can be commonly included in images acquired by different imaging devices (i.e., so that higher accuracy calibration between the respective imaging devices and the calibration reference frame can be achieved). Correspondingly, some implementations may include acquisition of multiple images of a calibration object with the object in different real-world locations or may include acquisition of one or more images with multiple calibration objects in different real-word locations (e.g., each with encoded coordinate information). In this way, for example, operators may more confidently ensure that particular features on the calibration object(s) are represented in associated images for multiple imaging devices (e.g., simultaneous images, or subsequent images without intervening movement of the relevant calibration object), and the disclosed system may thus produce more accurate relative calibration of an entire array of associated imaging devices.

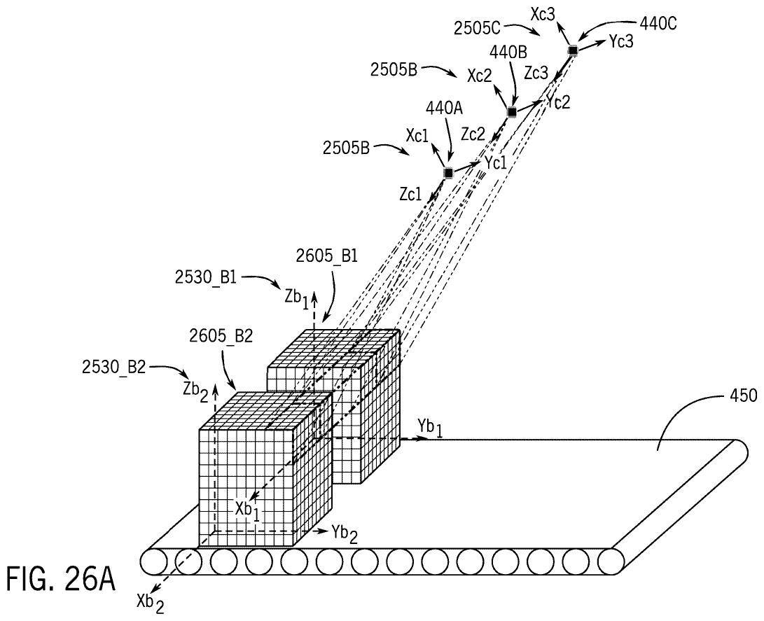

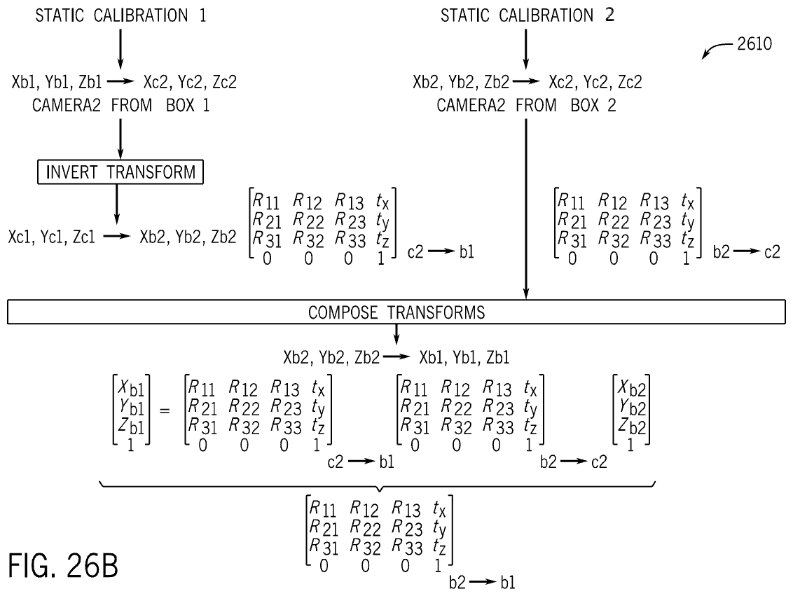

FIG. 26A illustrates an example configuration including three imaging devices (e.g., a first imaging device 440A, a second imaging device 440B, and a third imaging device 440C) in accordance with some embodiments of the technology. As illustrated in FIG. 26A, the imaging devices 440A, 440B, and 440C are associated, respectively, with a factory coordinate space (e.g., a first factory coordinate space 2505A, a second factory coordinate space 2505B, and a third factory coordinate space 2505C). In the illustrated example, the first imaging device 440A is associated with the first factory coordinate space 2505 (Xc1, Yc1, Zc1), the second imaging device 440B is associated with the second factory coordinate space 2505B (Xc2, Yc2, Zc2), and the third imaging device 440C is associated with the third factory coordinate space 2505C (Xc3, Yc3, Zc3).

FIG. 26A also illustrates two positions (e.g., a first position 2605_B1 and a second portion position 2605_B2) for the calibration target 2525, where each position is associated with a respective object coordinate space (e.g., a first object coordinate space 2530_B1 and a second object coordinate space 2530_B2). As illustrated in FIG. 26A, when the calibration target 2525 is in the first position 2605_B1, the calibration target 2525 is in the field of view of the first imaging device 440A and the second imaging device 440B. When the calibration target 2525 is in the second position 2605_B2, the calibration target 2525 is in the field of view of the second imaging device 440A and the third imaging device 440C. Accordingly, with respect to the first position 2605_B1 and the second position 2605_B2, the calibration target 2525 is in the field of view of the second imaging device 440B. Therefore, according to the illustrated example of FIG. 26A, the second imaging device 440B may be calibrated using a static calibration relative to the first position 2605_B1 and the second position 2605_B2 (e.g., as described in greater detail above with respect to FIGS. 25A-25C) and can thus be used to mathematically link the filed calibrations of the first and third imaging devices 440A, 440C.

In this regard, for example, FIG. 26B illustrates a process 2610 for relating the first object coordinate space 2530_B1 and the second object coordinate space 2530_B2 defined by the first position 2605_B1 and the second position 2605_B2 using a 3D rigid transformation (as generally described above in greater detail with respect to FIG. 25C). The 3D rigid transformations can then be composed to determine a transformation between the coordinate space 2530_B2 for the second position 2605_B2 and the coordinate space 2530_B1 for the first position 2605_B1. With sufficient such transformations having thus been determined (e.g., at least one for each pair of field calibration coordinate systems), the location of the various imaging devices relative to each other can be readily determined (e.g., as also discussed above).

Thus, for example, the process 2610 can result in all imaging devices (e.g., the first imaging device 440A, second imaging device 440B, and the third imaging device 440C) calibrated with respect to the object coordinate space 2530_B1 defined by the first position 2605_B1 of the calibration target 2525 (including imaging devices that don't have the calibration target 2525 in its field of view for that position, such as, e.g., the first imaging device 440A when the calibration target 2525 is in the second position 2605_B2).

Having all relevant imaging devices for a system calibrated with respect to a single coordinate space (defined by the calibration target 2525), or relative to multiple coordinate spaces with appropriately composed calibrations therebetween (see, e.g., FIG. 26B), can thus allow the relative location of the imaging devices to be determined. As also noted above, this determination of relative location can thus be used to assign names (e.g., identifiers) to the imaging device(s) and, for example, to verify that the system assembly matches a relevant design specification and has the proper coverage for the working volume. Further, if one or more of the calibration coordinate spaces can be calibrated to a relevant site coordinate system (e.g., by aligning a calibration object with the edge of the conveyor at a known origin location), similar approaches as discussed above can allow a system to automatically identify the imaging device(s) 440 positions and orientations with respect to the site (e.g., relative to the conveyor).

Although the example above considers multiple successive sets of image acquisitions with the calibration target 2525 in different locations, other approaches can utilize fewer (e.g., only one) set of simultaneous (or other) image acquisitions with multiple calibration targets (e.g., each substantially identical to the calibration target 2525) at different locations. For example, multiple calibration targets can be positioned so that a single image acquisition event for a tunnel or other system can result in acquired images that collectively include a sufficient number of commonly represented features among images for the various imaging devices so as to specify the relative (or absolute) location of all of the imaging devices.

In some embodiments, aspects of the technology, including computerized implementations of methods according to the technology, may be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, embodiments of the technology can be implemented as a set of instructions, tangibly embodied on a non-transitory computer-readable media, such that a processor device can implement the instructions based upon reading the instructions from the computer-readable media. Some embodiments of the technology can include (or utilize) a control device such as an automation device, a special purpose or general-purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the technology, or of systems executing those methods, may be represented schematically in the FIGs. or otherwise discussed herein. Unless otherwise specified or limited, representation in the FIGs. of particular operations in particular spatial order may not necessarily require those operations to be executed in a particular sequence corresponding to the particular spatial order. Correspondingly, certain operations represented in the FIGs., or otherwise disclosed herein, can be executed in different orders than are expressly illustrated or described, as appropriate for particular embodiments of the technology. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” “block,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g., “one or the other but not both”) when preceded by terms of exclusivity, such as “either,” “one of,” “only one of,” or “exactly one of.”

Also as used herein, unless otherwise limited or defined, the phrase “a set of” is intended to indicate a collection of elements, including a single element or multiple elements. For example, the phrase “a set of A” is intended to indicate “one or more of A” or “at least one of A.”

Although the present technology has been described by referring to preferred embodiments, workers skilled in the art will recognize that changes may be made in form and detail without departing from the scope of the discussion.

What is claimed is:

1. A method of commissioning an imaging device within a set of imaging devices of a machine vision system, the method comprising:
receiving commissioning data including a set of identifiers,
controlling the imaging device to capture image data of a calibration target;
determining, based on the captured image data, an identifier from the set of identifiers associated with the imaging device;
configuring the imaging device based on the determined identifier and the commissioning data; and
generating and transmitting a commissioning report for display to a user via a display device, the commissioning report indicating whether the imaging device was successfully configured.

2. The method of claim 1, wherein receiving the commissioning data includes receiving specification data identifying the set of imaging devices associated with the machine vision system and a technical setting for each imaging device.

3. The method of claim 1, further comprising:
generating and transmitting a commissioning details user interface for display to the user via the display device, the commissioning details user interface prompting the user to select a commissioning parameter; and
receiving a set of commissioning parameters based on user input provided via the commissioning details user interface, wherein the set of commissioning parameters includes at least one of a tunnel identifier, a site identifier, or an operator identifier.

4. The method of claim 1, further comprising:
generating and transmitting a methodology user interface for display to the user via the display device, the methodology user interface prompting the user to select a methodology parameter; and
receiving a set of methodology parameters based on user input provided via the methodology user interface, wherein the set of methodology parameters includes at least one of: a commissioning methodology, a material of the calibration target, a calibration target identifier, or a dimension of the calibration target.

5. The method of claim 4, wherein determining the identifier for the imaging device includes determining the identifier for the imaging device using the set of methodology parameters.

6. The method of claim 1, further comprising:
generating and transmitting a pre-commissioning checklist user interface for display to the user via the display device, wherein the pre-commissioning checklist user interface includes a set of pre-commissioning tasks to be performed prior to commissioning; and
receiving user confirmation that each pre-commissioning task included in the set of pre-commissioning tasks was completed.

7. The method of claim 1, wherein configuring the imaging device based on the identifier includes configuring the imaging device based on the identifier in response to receiving a user input confirming the association of the imaging device with the identifier.

8. The method of claim 1, further comprising:
generating and outputting an auto-naming user interface for display to the user via the display device, the auto-naming user interface indicating an identification status for each imaging device included in the set of imaging devices.

9. The method of claim 8, wherein generating and outputting the auto-naming user interface includes generating and outputting an auto-naming user interface indicating that a particular imaging device of the set of imaging devices was not associated with an identifier.

10. The method of claim 9, further comprising:
receiving a user-selected identifier for the particular imaging device based on user interaction with the auto-naming user interface, wherein the user-selected identifier is included in a list of remaining identifiers included in the auto-naming user interface.

11. The method of claim 10, wherein the list of remaining identifiers is generated based on identifiers that are not yet associated with any imaging device.

12. The method of claim 1, wherein each identifier of the set of identifiers is associated with at least one imaging device of the set of imaging devices.

13. A system for commissioning an imaging device within a set of imaging devices for a machine vision system, the system comprising:
at least one electronic processor configured to:
receive commissioning data including a set of identifiers
receive a set of commissioning parameters based on user input provided via a commissioning details user interface;
receive user confirmation based on user input provided via a pre-commissioning checklist user interface, the user confirmation confirming that each pre-commissioning task included in a set of pre-commissioning tasks was completed;
control the imaging device to capture image data of a calibration target;
determine, based on the captured image data, an identifier from the set of identifiers associated with the imaging device;
configure the imaging device based on the identifier and the commissioning data; and
generate and transmit a commissioning report for display to a user via a display device, the commissioning report indicating whether the imaging device of was successfully configured and including the set of commissioning parameters.

14. The system of claim 13, wherein each identifier of the set of identifiers is associated with at least one imaging device of the set of imaging devices.

15. A method of commissioning machine vision systems, the method comprising:
controlling acquisition of a plurality of images, including controlling a plurality of imaging devices to cause each imaging device of the plurality of imaging devices to capture an image of a calibration object, each of the imaging devices of the plurality of imaging devices having a factory calibration;
determining a field calibration for each imaging device of the plurality of imaging devices based on the image acquired by the imaging device; and
determining an updated calibration for each imaging device of the plurality of imaging devices based on the factory calibration and the field calibration for the imaging device.

16. The method of claim 15, further comprising:
determining an identifier for at least one imaging device of the plurality of imaging devices based on the updated calibration.

17. The method of claim 16, wherein the identifier is determined without calibrating the at least one imaging device of the plurality of imaging devices to an operational reference frame that includes the calibration object.

18. The method of claim 15, wherein capturing the image for each of the imaging devices of the plurality of imaging devices includes capturing the plurality of images such that the image of a first imaging device of the plurality of imaging devices and the image of a second imaging device of the plurality of the imaging devices include imaging data that represents a plurality of the same features on the calibration object, wherein the plurality of the same features includes a plurality of symbols on the calibration object that encode corresponding location information on the calibration object.

19. The method of claim 15, wherein the plurality of images includes a first plurality of images with the calibration object in a first location and a second plurality of images with the calibration object in a second location.

20. The method of claim 15, wherein the calibration object is a first calibration object and wherein the image for one or more of the imaging devices of the plurality of imaging devices includes the first calibration object and a second calibration object.

21. The method of claim 20, wherein determining the updated calibration based on the factory calibration and the field calibration includes determining a transform between calibrations for a plurality of the imaging devices based on the image that includes the first calibration object and the second calibration object.
